SEQUENCE LISTING (9 sequences)

SEQ ID NO: 1 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA

SEQ ID NO: 2 - protein, 771 aa, Homo sapiens [sequence omitted]

SEQ ID NO: 3 - protein, 749 aa, Homo sapiens
MGRAGAAAVIPGLALLWAVGLGSAAPSPPRLRLSFQELQAWHGLQTFSLERTCCYQALLVDEERGRLFVGAENHVASLNLDNISKRAKKLAWPAPVEWREECNWAGKDIGTECMNFVKLLHAYNRTHLLACGTGAFHPTCAFVEVGHRAEEPVLRLDPGRIEDGKGKSPYDPRHRAASVLVGEELYSGVAADLMGRDFTIFRSLGQRPSLRTEPHDSRWLNEPKFVKVFWIPESENPDDDKIYFFFRETAVEAAPALGRLSVSRVGQICRNDVGGQRSLVNKWTTFLKARLVCSVPGVEGDTHFDQLQDVFLLSSRDHRTPLLYAVFSTSSSIFQGSAVCVYSMNDVRRAFLGPFAHKEGPMHQWVSYQGRVPYPRPGMCPSKTFGTFSSTKDFPDDVIQFARNHPLMYNSVLPTGGRPLFLQVGANYTFTQIAADRVAAADGHYDVLFIGTDVGTVLKVISVPKGSRPSAEGLLLEELHVFEDSAAVTSMQISSKRHQLYVASRSAVAQIALHRCAAHGRVCTECCLARDPYCAWDGVACTRFQPSAKRRFRRQDVRNGDPSTLCSGDSSRPALLEHKVFGVEGSSAFLECEPRSLQARVEWTFQRAGVTAHTQVLAEERTERTARGLLLRRLRRRDSGVYLCAAVEQGFTQPLRRLSLHVLSATQAERLARAEEAAPAAPPGPKLWYRDFLQLVEPGGGGSANSLRMCRPQPALQSLPLESRRKGRNRRTHAPEPRAERGPRSATHW

SEQ ID NO: 4 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 5 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 6 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 7 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 8 - protein, 9 aa, Artificial Sequence
  gp100 peptide
KVPRNQDWL

SEQ ID NO: 9 - protein, 9 aa, Artificial Sequence
  Trp2 peptide
SVYDFFVWL

(19) United States
(12) Patent Application Publication
(10) Pub. No.: US 2022/0023342 A1
(43) Pub. Date: Jan. 27, 2022

(54) ANTI-NEUROPILIN-1 AND ANTI-PROGRAMMED CELL DEATH-1 COMBINATION THERAPY FOR TREATING CANCER

(71) Applicant: INSTITUT GUSTAVE ROUSSY, VILLEJUIF (FR)

(72) Inventors: FATHIA MAMI-CHOUAIB, BOURG LA REINE (FR); MARINE LECLERC, VILLEJUIF (FR); GEORGES BISMUTH, PARIS (FR)

(21) Appl. No.: 17/297,052

(22) PCT Filed: Dec. 2, 2019
(86) PCT No.: PCT/EP2019/083334

§ 371 Date: May 26, 2021

(30) Foreign Application Priority Data
Nov. 30, 2018 (EP) 18209675.0

(51) Int. Cl. A61K 35/17; C07K 16/28

(52) U.S. Cl. CPC A61K 35/17; A61K 2039/507; C07K 16/2866; C07K 16/2818

(57) ABSTRACT

The present invention relates to the combined use of a neuropilin-1 (Nrp-1) neutralizing agent and of a programmed cell death-1 (PD-1) neutralizing agent for killing cancer cells, typically for treating cancer, as well as to corresponding pharmaceutical compositions and kits, and to corresponding diagnostic and therapeutic methods. The invention further relates to in vitro, ex vivo and in vivo methods for detecting CD8⁺ TILs capable of recognizing cancer cells, for predicting the response of a subject to anti-PD-1 treatment of cancer, and for identifying a subject who responds therapeutically to a treatment of cancer with an antibody combination therapy comprising anti-Nrp-1 and anti-PD-1 antibodies.

### FIELD OF THE INVENTION

The present disclosure relates to the combined use of a neuropilin-1 (Nrp-1) neutralizing agent and of a programmed cell death-1 (PD-1) neutralizing agent (as checkpoint inhibitors) for killing cancer cells via immune cells, typically for treating cancer, as well as to corresponding pharmaceutical compositions and kits, and to corresponding diagnostic/predictive and therapeutic methods. The disclosure further relates to in vitro, ex vivo and in vivo methods for detecting CD8⁺ TILs capable of recognizing cancer cells, for predicting the response of a subject to anti-PD-1 treatment of cancer, and for identifying a subject who might respond therapeutically to a treatment of cancer with an antibody combination therapy comprising anti-Nrp-1 and anti-PD-1 antibodies.

### BACKGROUND OF THE INVENTION

Cytotoxic T lymphocytes (CTL), predominantly expressing T-cell co-receptor CD8, play a major role in the anti-tumor immune response. To destroy malignant cells, CTL must first migrate to the tumor site, infiltrate tumor cell clusters, and then interact with malignant cells to achieve their cytotoxic functions after T-cell receptor (TCR) recognition of specific tumor peptide-major histocompatibility complex class I (MHC-I) complexes on target cells¹. To perform this killing function, activated CTL are armed with various effector molecules, including pro-inflammatory cytokines, in particular IFNγ and TNFα, and cytotoxic granules containing perforin and granzymes. However, cancer cells frequently escape CD8⁺ T-cell recognition and reactivity. A decrease in cell surface expression of peptide/MHC-I complexes on cancer cells is one mechanism involved in this lack of functional activity. However, it now appears clear that engagement of inhibitory receptors such as cytotoxic T lymphocyte antigen-4 (CTLA-4), programmed cell death-1 (PD-1) and T cell immunoglobulin-3 (Tim-3), expressed on CD8⁺ tumor-infiltrating T lymphocytes (TIL) with their respective ligands on target cells, is another critical constraint explaining the poor reactivity of these cells in the tumor immune context². Moreover, PD-1 expression on CD8⁺ TIL appears to be a characteristic of clonally expanded CD8⁺ tumor-reactive T cells identified in cancer patients³.

In this context, elucidating the mechanisms of CTLA-4 and PD-1 T-cell inhibitory signalling has led to development of promising cancer immunotherapy tools, including blocking monoclonal antibodies (mAb) targeting these so-called “immune checkpoints”^{4,5}. Yet, to be efficient, immune checkpoint blockade therapies require strong tumor infiltration by CTL whose activities are subjected to such inhibition. Indeed, the therapeutic benefit of the PD-1 blockade requires that tumors be infiltrated by CD8⁺ TIL strongly expressing the PD-1 receptor⁶. Under these conditions, CD8⁺ T-cell responses to tumor-specific antigens parallel tumor regression, and appear to be directly associated with clinical benefits of anti-PD-1 immunotherapy^{3,7}. Unfortunately, only a fraction of cancer patients responds to PD-1 blockade and, among long-term responders, relapses are often observed after initial tumor regression. In this regard, a high proportion of tumors, referred to as “immune deserts” or “cold tumors”, are not infiltrated by immune cells and are thus poorly responsive to immunotherapy. Moreover, additional as yet undescribed inhibitory mechanisms inherent to CD8⁺ TIL and leading to tumor resistance likely exist.

In the context of the present invention, inventors analysed the role of neuropilin-1 (Nrp-1) during anti-tumor CD8⁺ T-cell responses. Nrp-1 acts as a co-receptor for extracellular ligands, including secreted members of the class 3 semaphorin (Sema-3) family, isoforms of vascular endothelial growth factor (VEGF) and transforming growth factor (TGF)β⁸. This transmembrane glycoprotein is involved in axon guidance and neuronal development^{9,10}. In mice, Nrp-1 is expressed at high levels on natural regulatory T (nTreg) cells, and its expression by Treg cells is responsible for reduced anti-tumor immunity^{11,12}. Nrp-1 deficiency on murine Foxp-3⁺ CD4⁺ Treg cells has been reported to delay tumor growth correlated with enhanced activation of tumor-infiltrating CD8⁺ T cells¹³. In humans, Nrp-1 is expressed on a subset of CD4⁺ Treg cells in lymph nodes¹⁴ and on CD4⁺ TIL, including suppressive Treg¹⁵. However, much less is known about the expression of Nrp-1 on CD8⁺ TIL and any possible involvement of Nrp-1 in regulating their functions. Nrp-1 has been found to be expressed by murine-tolerant self-reactive CD8⁺ T cells, and by human melanoma-infiltrating CD8⁺ T lymphocytes¹⁶. Nevertheless, the role of Nrp-1 on CD8⁺ TIL in inhibiting anti-tumor T-cell functions was unknown up to the present invention.

### SUMMARY OF THE INVENTION

Neuropilin-1 (Nrp-1) has been defined as a marker of murine CD4⁺Foxp3⁺ regulatory T (Treg) cells and a subset of human CD4⁺ Treg cells. It is also expressed by a population of CD8⁺ T cells infiltrating some solid tumors. However, little is known about its contribution to regulation of tumor-specific CD8⁺ T-cell functions. Inventors herein reveal that Nrp-1 defines a subset of CD8⁺ T cells displaying PD-1^{high} (PD-1^{hi}) status and infiltrating cancer cells. Using a human cytotoxic T lymphocyte (CTL) clone model, they showed that the interaction of Nrp-1 with its ligand semaphorin-3A (Sema-3A), secreted by autologous human non-small cell lung cancer (NSCLC) cells, inhibits both CTL migration and tumor-specific lytic function. In vivo experiments in mouse models revealed that this Nrp-1⁺ PD-1^{hi} CD8⁺ tumor-infiltrating T lymphocyte (TIL) subset is also found in engrafted B16F10 mouse melanoma, and is enriched with tumor-specific T cells exhibiting an exhausted state, with co-expression of Tim-3, LAG-3 and CTLA-4 inhibitory receptors. Functionally, anti-Nrp-1 neutralizing antibodies increase the migratory capacity of Nrp⁺ PD-1^{hi} CD8⁺ TILs toward autologous cancer cells and enhance their specific cytotoxic activity ex vivo. Remarkably, in vivo immunotherapeutic blockade of Nrp-1 results in control of tumor growth, with a parallel increase in tumor infiltration by CD8⁺ TILs and enhanced proliferative and cytotoxic potential. This blockade is cumulative with anti-PD-1 treatment in improving tumor regression.

Inventors herein describe for the first time Nrp-1 as a novel immune checkpoint target on CD8⁺ tumor-specific T cells for cancer immunotherapies, in particular for combined antibody-based cancer immunotherapies.

The present invention provides improved tools and methods for enhancing an anti-tumor immune response via the combined use in particular of neuropilin-1 (Nrp-1) and programmed cell death-1 (PD-1) neutralizing agents, for example via the combined use of anti-Nrp-1 and anti-PD-1 antibodies.

In one aspect, inventors herein describe a combination comprising an effective amount of a neuropilin-1 (Nrp-1) neutralizing agent, for example an anti-Nrp-1 antibody, and an effective amount of a programmed cell death-1 (PD-1) neutralizing agent, for example an anti-PD-1 antibody, for separate, concurrent or sequential use in killing cancer cells, in particular for use in treating cancer in a subject in need thereof. This combination of a Nrp-1 neutralizing agent and of a PD-1 neutralizing agent is particularly advantageous for use in activating CD8⁺ tumor-infiltrating T lymphocytes (TILs) anti-cancer cell effector functions and/or for enhancing cancerous tumor infiltration by CD8⁺ TILs having active anti-cancer cells effector functions, in particular PD-1⁺ CD8⁺ TILs, preferably Nrp-1³⁰ PD-1⁺ CD8⁺ TILs.

In other aspects, inventors herein describe a pharmaceutical composition comprising a Nrp-1 neutralizing agent, a PD-1 neutralizing agent, and a pharmaceutically acceptable carrier, as well as a kit comprising i) a Nrp-1 neutralizing agent and ii) a PD-1 neutralizing agent, in different containers.

Also, herein described is a population of Nrp-1⁺ CD8⁺ tumor-infiltrating T lymphocytes (TILs) exhibiting anti-cancer cell effector functions and/or enhanced cancerous tumor infiltration ability for use in treating a cancer in a subject. In a preferred aspect, the population of TILs has been obtained from the subject to be treated and TILs have been amplified ex vivo. In another preferred aspect, Nrp-1 CD8⁺ TILs are PD-1^{T} TILs.

Also, herein described is an in vitro, ex vivo or in vivo method for detecting CD8⁺ TILs capable of recognizing cancer cells, in particular PD-1^{T} TILs. This method typically comprises a) providing a biological sample comprising TILs and b) determining i) whether said TILs express Nrp-1, and optionally ii) whether said TILs express one or several receptors selected from CTLA-4, Tim-3 and LAG-3 and/or iii) the PD-1⁺ status of said TILs, the expression of Nrp-1 by the TILs indicating that said TILs are capable of recognizing cancer cells.

Further herein described is an in vitro, ex vivo or in vivo method for predicting the response of a subject to anti-PD-1 treatment of cancer. This (predictive) method typically comprises detecting CD8⁺ TILs recognizing cancer cells as herein described, the presence in a biological sample of the subject of TILs expressing Nrp-1, in particular of Nrp-1⁺ PD-1⁺ CD8⁺ TILs, being indicative that the subject does not (i.e. would not as the method is a predictive method) respond, or does not (i.e. would not as the method is a predictive method) optimally respond, to the anti-PD-1 treatment of cancer.

Also described is an in vitro, ex vivo or in vivo (predictive) method for identifying a subject who (would) respond therapeutically to a treatment of cancer with an antibody combination therapy comprising an effective amount of a Nrp-1 neutralizing antibody and an effective amount of a PD-1 neutralizing antibody. This method typically comprises detecting CD8⁺ TILs recognizing cancer cells as described in the herein described corresponding method, the presence in a biological sample of the subject of TILs expressing Nrp-1, in particular of Nrp-1⁺ PD-1⁺ CD8⁺ TILs, being indicative that the subject responds (would respond) to the antibody combination therapy.

Also, herein described is an in vitro, ex vivo or in vivo method for predicting the response of a human subject to a treatment of lung cancer combining anti-PD-1 and anti-Nrp-1 agents, the method comprising a step of detecting CD8⁺ TILs recognizing cancer cells, the presence, in the TILs of a biological sample of the subject, of about 15±5% of Nrp-1⁺ PD-1⁺ CD8⁺ TILs being indicative that the human subject respond to the treatment of cancer.

### DETAILED DESCRIPTION OF THE INVENTION

Neuropilin-1 (Nrp-1 or NRP1) is a protein that in humans is encoded by the NRP1 gene. In humans, the NEUROPILIN 1 gene is located at 10p11.22. This is one of two human neuropilins. Neuropilin 1 is a membrane-bound coreceptor to a tyrosine kinase receptor for both vascular endothelial growth factor (VEGF; MIM 192240) and semaphorin (see SEMA3A; MIM 603961) family members. Nrp-1 plays versatile roles in angiogenesis, axon guidance, cell survival, migration, and invasion. Nrp-1 has been implicated in the vascularization and progression of cancers. Nrp-1 expression has been shown to be elevated in a number of human patient tumor samples, including brain, prostate, breast, colon, and lung cancers and Nrp-1 levels are positively correlated with metastasis.

In prostate cancer (PCa), NRP1 has been demonstrated to be an androgen-suppressed gene, upregulated during the adaptive response of prostate tumors to androgen-targeted therapies and a prognostic biomarker of clinical metastasis and lethal PCa. In vitro and in vivo mouse studies have shown membrane bound Nrp-1 to be proangiogenic and that Nrp-1 promotes the vascularization of prostate tumors. Elevated Nrp-1 expression is also correlated with the invasiveness of non-small cell lung cancer (NSCLC) both in vitro and in vivo.

“NRP1” refers to any variant, derivative, or isoform of the NRP1 human gene.

“Nrp-1” refers to any variant, derivative, or isoform of the protein of SEQ ID NO: 1 encoded by the NRP1 human gene.

Natural ligands for Nrp-1 have been identified: Sema-3A, Sema-3B and VEGF A.

The complete human Sema-3A sequence can be found under UniProtKB/Swiss-Prot, identifier: Q14563 (SEQ ID NO: 2).

The complete human Sema-3B sequence can be found under UniProtKB/Swiss-Prot, identifier: Q13214 (SEQ ID NO: 3).

The complete human VEGF A sequence can be found under UniProtKB/Swiss-Prot, identifier: P15692 (SEQ ID NO: 4).

In the context of the present invention, inventors demonstrated that Nrp-1 is expressed on a subset of human CD8⁺ TIL in non-small-cell lung cancer (NSCLC) tumors and on murine CD8⁺ TIL from B16F10 melanoma. These T lymphocytes also express high levels of PD-1, defining a new subset of Nrp-1⁺ PD-1^{hi} TIL enriched with tumor-specific T cells and displaying an exhausted state. Importantly, inventors showed that the interaction of human Nrp-1 with its ligand Sema-3A (semaphorin-3A) inhibits T-cell migration and tumor-specific TCR-mediated cytotoxic functions in vitro. In contrast, anti-Nrp-1 neutralizing antibodies increase the migratory capacity of Nrp-1⁺ PD-1⁺ CD8⁺ TILs, in particular of Nrp-1⁺ PD-1^{hi} CD8⁺ TILs, toward autologous cancer cells and enhance their specific cytotoxic activity ex vivo. In addition, inventors showed that anti-Nrp-1 blocking mAb restore CD8⁺ T-cell effector functions and optimize control of tumor growth induced by anti-PD-1 in vivo. Inventors herein reveal that Nrp-1 negatively regulates anti-tumor CD8⁺ T-cell immunity and is thus an advantageous target for combined cancer immunotherapies.

In the context of the present invention, “Nrp-1 positive lymphocyte or T-cell” or “Nrp-1⁺ lymphocyte or T-cell” generally refers to T-cells expressing Nrp-1 on the cell-surface, typically CD8⁺ T cells, preferably CD8⁺ tumor infiltrating lymphocytes (CD8⁺ TILs), which can be detected by e.g. flow-cytometry using antibodies that specifically recognize a Nrp-1 epitope. “Nrp-1⁺ lymphocyte or T-cell” also includes T cell lines and clones.

Particular “Nrp-1⁺ lymphocytes or T-cells” are Nrp-1⁺ PD-1⁺ CD8⁺ TILs, for example TILs expressing a high level of PD-1 (PD-1^{high} or PD-1^{hi} TILs) as do PD-1^{T} lymphocytes as described by Thommen et al.³⁵.

“PD-1” refers to the protein Programmed Death 1 (PD-1) (also referred to as “Programmed Cell Death 1”), an inhibitory member of the CD28 family of receptors, that also includes CD28, CTLA-4, ICOS and BTLA. PD-1 is expressed on activated B cells, T cells, and myeloid cells Okazaki et al. (2002) Curr. Opin. Immunol. 14: 391779-82; Bennett et al. (2003) J Immunol 170:711-8). The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260).

“PD1” refers to any variant, derivative, or isoform of the PD1 human gene.

“PD-1” refers to any variant, derivative, or isoform of the protein of SEQ ID NO: 5 encoded by the PD1 human gene. The complete human PD-1 aa sequence (SEQ ID NO: 5) can be found under GenBank Accession No. U64863.

Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members.

The complete human PD-L1 sequence can be found under UniProtKB/Swiss-Prot, identifier Q9NZQ7-1 (SEQ ID NO: 6).

The complete human PD-L2 sequence can be found under UniProtKB/Swiss-Prot, identifier: Q9BQ51 (SEQ ID NO: 7).

In the context of the present invention, “PD-1 positive lymphocyte or T-cell” or “PD-1⁺ lymphocyte or T-cell” generally refers to T-cells expressing PD-1 on the cell-surface, typically CD8⁺ T cells, preferably CD8⁺ tumor infiltrating lymphocytes (CD8⁺ TILs), which can be detected by e.g. flow-cytometry using antibodies that specifically recognize a PD-1 epitope. “PD-1⁺ lymphocyte or T-cell” also includes T cell lines or clones.

Particular “PD-1⁺ lymphocytes or T-cells” are TILs expressing a high level of PD-1 (PD-1^{high} or PD-1^{hi} TILs) such as PD-1^{T} lymphocytes as described by Thommen et al.³⁵ (having an intrinsically high capacity for tumor recognition). Particular “PD-1⁺ lymphocytes or T-cells” are Nrp-1⁺ PD-1⁺ CD8⁺ TILs. Particular “PD-1⁺ lymphocytes or T-cells” are “PD-1^{high} lymphocytes or T-cells” (PD-1^{T}).

Particular “PD-1^{high} lymphocytes or T-cells” (PD-1^{T}) are Nrp-1⁺ PD-1⁺ CD8⁺ TILs.

Inventors thus herein describe for the first-time a (pharmaceutical) combination for use in the context of an immunotherapy comprising an effective amount of a neuropilin-1 (Nrp-1) neutralizing agent and an effective amount of a programmed cell death-1 (PD-1) neutralizing agent for in vitro, ex vivo or in vivo separate, concurrent or sequential use in killing cancer cells, typically for killing cancer cells or treating cancer in a subject in need thereof.

The term “subject” refers to any subject, in particular a mammal.

Examples of mammals include humans and non-human animals such as, without limitation, domesticated animals (e.g., cows, sheep, cats, dogs, and horses), non-human primates (such as monkeys), rabbits, and rodents (e.g., mice and rats). The treatment is preferably intended for a human being in need thereof, whatever its age or sex.

The term “subject” typically designates a patient, in particular a patient having a cancer or tumor. Unless otherwise specified in the present disclosure, the tumor is a cancerous or malignant tumor. In a particular aspect, the subject is a subject undergoing a treatment of cancer such as chemotherapy and/or radiotherapy, or a subject at risk, or suspected to be at risk, of developing metastasis.

The subject is, for example a human being suffering of a cancer and resistant to cancer treatment, typically to chemotherapeutic agents including checkpoint blockers.

The subject may have been exposed to part of a complete conventional treatment protocol, for example to at least one cycle of the all treatment protocol, for example two cycles of the all treatment protocol, the treatment including typically anti-PD-1 agent(s), for example a combination comprising a PD-1 neutralizing agent and a Nrp-1 neutralizing agent.

The subject is preferably a subject, in particular a human subject, having tumor infiltrating lymphocytes (TILs), typically CD8⁺ TILs, in particular PD-1⁺ CD8⁺ TILs, preferably PD-1^{high} (PD-1^{hi}) CD8⁺ TILs such as PD-1^{T} lymphocytes as described by Thommen et al.³⁵ (having an intrinsically high capacity for tumor recognition) and/or Nrp-1⁺ CD8⁺ TILs. In a preferred aspect, the subject is a subject, preferably a human subject having Nrp-1⁺ PD-1⁺ CD8⁺ TILs.

The subject is typically a subject that would benefit from administration of a combination of a Nrp-1 and of a PD-1 neutralizing agent, for example a combination of antibodies and of any additional therapeutic agent(s) or composition(s) of interest.

The cancer or tumor may be any kind of cancer or neoplasia. The cancer can be a metastatic cancer or primary cancer.

The tumor is typically a solid tumor, in particular of epithelial, neuroectodermal or mesenchymal origin.

In a particular aspect, the cancer is a solid tumor, preferably selected from a carcinoma, a sarcoma and a blastoma. In an aspect, the solid tumor is an advanced and/or refractory tumor.

In a preferred aspect, the cancer is a carcinoma, for example an adenocarcinoma, preferably a lung cancer including non-small-cell lung cancer (NSCLC) and small-cell lung cancer (SCLC), in particular a non-small cell lung cancer (NSCLC), or a breast or colon cancer.

In another preferred aspect, the carcinoma is a melanoma.

In another particular aspect, the cancer is a hematological tumor, preferably selected from lymphoma, leukemia and multiple myeloma.

In a particular aspect, the cancer is a leukemia, typically an acute myelogenous leukemia (AML) or a chronic lymphocytic leukemia.

In another particular aspect, the cancer is a colon cancer, typically a colon carcinoma. The cancer may also be a colorectal cancer.

In another particular aspect, the cancer is a breast or a prostate cancer.

In a further particular aspect, the cancer is a brain cancer.

In another aspect, the cancer is a pediatric cancer typically a pediatric sarcoma, lymphoma, leukemia, neuroblastoma, brain cancer, or central nervous system cancer.

In the context of the present invention, cancer cells are cells from any one of the herein above described cancer, in particular cells selected from the group consisting of lung cancer, in particular non-small-cell lung cancer (NSCLC), melanoma, renal cancer, colorectal cancer, breast cancer, prostate cancer, brain cancer, soft tissue sarcoma, pancreatic cancer, neuroendocrine tumors or thyroid carcinoma, cells.

Inhibition or neutralization of Nrp-1, preferably of the inhibitory activity of Nrp-1 towards anti-tumor immune response, can advantageously involve use of a polypeptide (e.g. an antibody, a fragment, variant, derivative or analog thereof, typically an antigen binding fragment thereof that specifically binds Nrp-1, a polypeptide fused to an Fec domain, etc.) that prevents Nrp-1 ligand-induced Nrp-1 signalling, e.g. by blocking the interaction with its natural ligand(s) Sema-3A, Sema-3B and/or vascular endothelial growth factor A (VEGF A).

In one aspect, the polypeptide is a protein kinase inhibitor such as LKB1 [cf. Okon, I. S. et al. Protein kinase LKB1 promotes RAB7-mediated neuropilin-1 degradation to inhibit angiogenesis. J Clin Invest 124, 4590-4602 (2014)] or imatinib [Raimondi, C. et al. Imatinib inhibits VEGF-independent angiogenesis by targeting neuropilin 1-dependent ABL1 activation in endothelial cells. J Exp Med 211, 1167-1183 (2014)].

In second aspect, the polypeptide is an antibody that binds Nrp-1 (an “anti-Nrp-1 antibody”). Such antibody may block the interaction between Nrp-1 and Sema-3A, between Nrp-1 and Sema-3B and/or between Nrp-1 and VEGF A. In another aspect, the polypeptide is an antibody that binds Sema-3A (an “anti-Sema-3A antibody”), Sema-3B (an “anti-Sema-3B antibody”) and/or VEGF A (an “anti-VEGF A antibody”) and blocks the interaction between Nrp-1 and Sema-3A, Sema-3B and/or VEGF A.

Inhibition or neutralization of PD-1, preferably of the inhibitory activity of PD-1 towards anti-tumor immune response, can advantageously involve use of a polypeptide (e.g. an antibody, a fragment, variant, derivative or analog thereof, typically an antigen binding fragment thereof that specifically binds PD-1, a polypeptide fused to an Fc domain, an immunoadhesin, etc.) that prevents PD-L1-induced PD-1 signalling, e.g. by blocking the interaction with its natural ligand PD-L1 (and optionally further blocking the interaction between PD-1 and PD-L2) or that prevents PD-L2-induced PD-1 signalling, e.g. by blocking the interaction with its natural ligand PD-L2 (and optionally further blocking the interaction between PD-1 and PD-L1). In one aspect the polypeptide is an antibody that binds PD-1 (an “anti-PD-1 antibody”). Such antibody may block the interaction between PD-1 and PD-L1 and/or between PD-1 and PD-L2. In another aspect, the polypeptide is an antibody that binds PD-L1 (an “anti-PD-L1 antibody”) and blocks the interaction between PD-1 and PD-L1. In another aspect, the polypeptide is an antibody that binds PD-L2 (an “anti-PD-L2 antibody”) and blocks the interaction between PD-1 and PD-L2.

The inhibitory activity of a polypeptide towards anti-tumor immune response refers to a process in which the polypeptide is inhibited in its capacity to negatively affect intracellular processes leading to lymphocyte responses such as cytokine release and cytotoxic responses. This can be measured for example in a T-cell based cytotoxicity assay, in which the capacity of a therapeutic compound to stimulate killing of tumor cells by lymphocytes, typically by Nrp-1⁺ PD-1⁺ CD8⁺ TILs, is measured. In one embodiment, an antibody preparation causes at least a 10% augmentation in the cytotoxicity of the lymphocytes, optionally at least a 40% or 50% augmentation in lymphocytes cytotoxicity, optionally at least a 70% augmentation in lymphocytes cytotoxicity”. If a neutralizing agent blocks the interaction between the polypeptide and its ligand(s), it typically (re)activates or increases the cytotoxicity of lymphocytes expressing said polypeptide. This can be evaluated for example via a standard in vitro cytotoxicity assay such as chromium release assay. Activation or increase of T cell cytotoxicity can be assessed for example by measuring (an increase of) cytokine production (for example IFN-7 production) or cytotoxicity surface markers (for example CD107a). CD107a, also known as lysosomal-associated membrane protein-1 (LAMP-1), is used as a marker of CD8⁺ T-cell degranulation following stimulation. Cytokine production from cells may be assessed for example by intracytoplasmic staining and analysis by flow cytometry after several days in culture, or using ELISA or Elispot assay.

As used herein, the terms “polypeptide,” “peptide” and “protein” are used interchangeably herein and refer to a molecule composed of monomers (amino acids, “aa”) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain”, or any other term used to refer to a chain or chains of two or more amino acids, are included within the definition of “polypeptide”, and the term “polypeptide” may be used instead of, or interchangeably with any of these terms. The term “polypeptide” is also intended to refer to the products of post-expression modifications of the polypeptide, whether natural or non-natural (artificial), including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “antibody” and “immunoglobulin” are used interchangeably herein. An antibody or immunoglobulin comprises at least the variable domain of a heavy chain, and normally comprises at least the variable domains of a heavy chain and a light chain. The terms variable light chain (V_{L}) and variable heavy chain (V_{H}) refer to these light and heavy chains respectively. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The V_{L} domain and V_{H} domain, or subset of the complementarity determining regions (CDRs), of an antibody combine to form the variable region that defines a three-dimensional antigen binding site. More specifically, the antigen binding site is defined by three CDRs on each of the V_{L} and V_{H} chains. In some instances, e.g., certain immunoglobulin molecules derived from camelid species or engineered based on camelid immunoglobulins, a complete immunoglobulin molecule may consist of heavy chains only, with no light chains.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., 71-74). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies or antigen-binding fragments, variants, or derivatives thereof for use in the context of the invention include, but are not limited to, polyclonal, monoclonal (“mAb”), multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a V_{L} or V_{H} domain, fragments produced by an Fab expression library, and anti-idiotypic (anti-Id) antibodies (including, e.g., anti-Id antibodies to Nrp-1 or PD-1 antibodies disclosed herein), or otherwise human-suitable antibodies. ScFv molecules, for example, are known in the art and are described, e.g., in U.S. Pat. No. 5,892,019. Immunoglobulin or antibody molecules used in the context of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

The term “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor.

Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term “conformational” is therefore often used interchangeably with “structural”.

The terms “fragment”, “variant”, “derivative” and “analog” when referring to polypeptides or antibodies (also herein identified as antibody polypeptides) of the present invention include any polypeptides which retain at least some of the neutralizing activity and/or of the antigen-binding properties of the corresponding native polypeptide or antibody.

Antibody fragments, including single-chain antibodies, may comprise the variable region(s) alone or in combination with the entirety or a portion of the following: hinge region, CH1, CH2, and CH3 domains.

Also included in the invention are antigen-binding fragments also comprising any combination of variable region(s) with a hinge region, CH1, CH2, and CH3 domains. Fragments of polypeptides of the present invention include proteolytic fragments, as well as deletion fragments, in addition to specific antibody fragments herein described.

Variants of Nrp-1 and/or PD-1 antibodies and antibody polypeptides of the present invention include fragments as described above, and also polypeptides with altered amino acid sequences due to amino acid substitutions, deletions, or insertions. Variants may occur naturally or be non-naturally occurring Non-naturally occurring variants may be produced using art-known mutagenesis techniques. Variant polypeptides may comprise conservative or non-conservative amino acid substitutions, deletions or additions. Variants of the antibodies include humanized versions of the antibodies as well as antibodies that have been affinity matured or optimized. Affinity optimization can be performed by routine methods that are well-known in the art. Alternatively, a preferred method for increasing the affinity of antibodies of the invention is disclosed in US 2002/0123057 A1.

Derivatives of Nrp-1 and/or PD-1 antibodies and antibody polypeptides of the present invention, are polypeptides which have been altered so as to exhibit additional features not found on the native polypeptide. Examples include fusion proteins. As used herein a “derivative” of a Nrp-1 and/or PD-1 antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In one aspect of the disclosure, the Nrp-1 neutralizing agent and/or the PD-1 neutralizing agent is a chimeric, (fully) human or humanized antibody, or a fragment thereof that binds to, preferably that specifically or preferentially binds to, and neutralize, the Nrp-1 polypeptide, and/or a fragment thereof that binds to, preferably that specifically or preferentially binds to, and neutralize the PD-1 polypeptide.

By “specifically binds”, it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope.

As used herein, the term “affinity” refers to a measure of the strength of the binding of an individual epitope with the CDR of an immunoglobulin molecule [see, e.g., Harlow et al., Antibodies: A Laboratory Manual, (Cold Spring Harbor Laboratory Press, 2nd ed. 1988) at pages 27-28]. The affinity of an antibody is given by the dissociation constant Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Competitive binding assays and other methods for determining specific binding are well known in the art. For example, binding can be detected via radiolabels, physical methods such as mass spectrometry, or direct or indirect fluorescent labels detected using, e.g., cytofluorometric analysis (e.g. FACScan). Binding above the amount seen with a control, non-specific agent indicates that the agent binds to the target.

By “preferentially binds”, it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

In the context of the present invention, a “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The terms “Fc domain,” “Fc portion,” and “Fc region” refer to a C-terminal fragment of an antibody heavy chain, e.g., from about amino acid (“aa”) 230 to about aa 450 of human γ (gamma) heavy chain or its counterpart sequence in other types of antibody heavy chains (e.g., α, δ, ε and μ for human antibodies), or a naturally occurring allotype thereof.

A “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge [see, e.g., Green et al. (1994) Nature Genet 7:13; Lonberg et al. (1994) Nature 368:856; Taylor et al. (1994) Int Immun 6:579, the entire teachings of which are herein incorporated by reference]. A fully human antibody also can be constructed by genetic or chromosomal transfection methods, as well as phage display technology, all of which are known in the art [see, e.g., McCafferty et al. (1990) Nature 348:552-553]. Human antibodies may also be generated by in vitro activated B cells [see, e.g., U.S. Pat. Nos. 5,567,610 and 5,229,275, which are incorporated in their entirety by reference].

In a particular aspect, the Nrp-1 neutralizing antibody comprises the CDR1, CDR2 and CDR3 domains of a heavy chain, and the CDR1, CDR2 and CDR3 domains of a light chain of an available antibody known by the skilled person.

Another exemplary Nrp-1 neutralizing antibody comprises a sequence having amino acid sequence identity of at least 50%, 60%, 70%, 80%, 90%, 95%, 98% or 99% with any one of such CDR1, CDR2 and CDR3 of known heavy and light chains, an antigen-binding fragment or a variant thereof.

For example, a human Nrp-1 neutralizing antibody can be selected from the group consisting of any known human Nrp-1 neutralizing antibodies, and can be for example YW64.3 or YW107.4.87 [Liang, W. C. et al. Function blocking antibodies to neuropilin-1 generated from a designed human synthetic antibody phage library. J Mol Biol 366, 815-829 (2007)], and a variant or a derivative thereof that retain the neutralizing activity and the binding specificity for Nrp-1.

Further known Nrp-1 antibodies and other Nrp-1 neutralizing agents include anti-Sema3A mAb, anti-Sema-3B mAb and anti-VEGF A mAb.

An example of a mouse Nrp-1 neutralizing antibody can be selected from MAB59941 (R&D system; Delgoffe G M et al., Nature. 2013 Sep. 12; 501(7466):252-6. Stability and function of regulatory T cells is maintained by a neuropilin-1-semaphorin-4a axis), and variants or derivatives thereof that retain the neutralizing activity and the binding specificity for Nrp-1.

In a particular aspect, the PD-1 neutralizing antibody comprises the CDR1, CDR2 and CDR3 domains of a heavy chain, and the CDR1, CDR2 and CDR3 domains of a light chain of an available antibody known by the skilled person.

Another exemplary PD-1 neutralizing antibody comprises a sequence having an amino acid sequence identity of at least 50%, 60%, 70%, 80%, 90%, 95%, 98% or 99% with any one of such CDR1, CDR2 and CDR3 of known heavy and light chains, an antigen-binding fragment or a variant thereof.

For example, a human PD-1 neutralizing antibody can be selected from the group consisting of any known human PD-1 neutralizing antibodies, and can be for example selected from the group consisting of nivolumab (Opdivo® from Bristol-Myers Squibb), lambrolizumab or pembrolizumab (Keytruda® from Merk), Pidlizumab (CT-011; CureTech), and a variant or a derivative thereof that retain the neutralizing activity and the binding specificity for PD-1.

Further known PD-1 antibodies and other PD-1 neutralizing agents include anti-PD-L1 mAb such as Avelumab (BAVENCIO® anti-PD-L1 from Merck), Atezolizumab (Tecentriq® anti-PD-L1 from Genentech) and Durvalumab (Imfinzi from Medimmune/AstraZeneca), and anti PD-L2 mAb.

A drug CA-170 (under clinical trials) that directly targets the Programmed death-ligands 1 and 2 (PD-L1/PD-L2), and V-domain Ig suppressor of T cell activation (VISTA) immune checkpoints (from Curis Inc) may also be used.

MPDL3280A/RG7446 (anti-PD-L1 from Roche/Genentech) is a human anti-PD-L1 mAb that contains an engineered Fc domain designed to optimize efficacy and safety by minimizing FcγR binding and consequential antibody-dependent cellular cytotoxicity (ADCC).

A mouse PD-1 neutralizing antibody can be selected from the group consisting of 29F.1A12 (Bio-X-Cell), RMP1-14 (Bio-X-Cell) or J43 (Bio-X-Cell), and variants or derivatives thereof that retain the neutralizing activity and the binding specificity for PD-1.

Also, herein described are Nrp-1⁺ CD8⁺ tumor-infiltrating T lymphocytes (TILs) for use in treating a cancer in a subject, in particular a therapeutic composition comprising Nrp-1⁺ CD8⁺ TILs, typically a population of PD-1⁺ CD8⁺ TILs, said Nrp-1⁺ CD8⁺ TILs exhibiting anti-cancer cell effector functions and/or enhanced cancerous tumor infiltration ability, in particular an enhanced cancerous (tumor) T-cell infiltration ability.

Preferred Nrp-1⁺ CD8⁺ TILs exhibiting enhanced cancerous tumor infiltration ability are activated CD3⁺ CD8⁺ TILs.

In a preferred aspect, these TILs have been obtained from the subject to be treated and have been amplified ex vivo. According to a particular aspect of the invention, Nrp-1⁺ TILs are isolated by FACS using an anti-Nrp-1 antibody, and then amplified in vitro, for example on irradiated allogenic feeder cells as described in the art [Chandran S S et al., Lancet Oncol. 2017 June; 18(6):792-802. Parkhurst M, Clin Cancer Res. 2017 May 15; 23(10):2491-2505. Echchakir, H et al, Int Immunol. 2000 April; 12(4):537-46].

In a preferred aspect, Nrp-1⁺ CD8⁺ TILs are PD-1^{T} TILs.

The therapeutic composition comprising Nrp-1⁺ CD8⁺ TILs may in addition comprise a pharmaceutically acceptable carrier or support, typically a cell culture medium, preferably adapted to the culture or preservation of lymphocytes such as complete RPMI media supplemented with recombinant IL-2 [cf. Chandran S S et al., Lancet Oncol. 2017 June; 18(6):792-802. Parkhurst M, Clin Cancer Res. 2017 May 15; 23(10):2491-2505. Echchakir, H et al, Int Immunol. 2000 April; 12(4):537-46].

In a particular aspect, the cancer is a solid tumor, preferably selected from a carcinoma, in particular a lung cancer [for example a non-small cell lung cancer (NSCLC)], or a melanoma; a sarcoma; and a blastoma. In another particular aspect, the cancer is a hematological tumor, preferably selected from lymphoma, leukemia and multiple myeloma.

As used herein, “an effective amount or dose” or “a therapeutically effective amount or dose” refers to an amount of the compound or composition of the invention which removes, slows down the disease, in particular the cancer, or reduces or delays one or several symptoms or disorders caused by or associated with said disease in the subject, or which induce a measurable immune response in the subject, who is preferably a human being. The effective amount, and more generally the dosage regimen, of the compounds, typically of the neutralizing agents, and pharmaceutical compositions, including pharmaceutical compositions thereof, of the invention, may be determined and adapted by the one skilled in the art. An effective dose can be determined by the use of conventional techniques and by observing results obtained under analogous circumstances. The therapeutically effective dose of the compounds and compositions of the invention will vary depending on the disease to be treated, its gravity, the route of administration, any co-therapy involved, the patient's age, weight, general medical condition, medical history, etc.

An effective amount of a neutralizing agent, for example an antibody, which is part of a (pharmaceutical) combination according to the present invention is typically an amount effective for the (pharmaceutical) combination to kill cancer cells in vitro, ex vivo or in vivo when in contact with said cells or when administered to a subject comprising said cells.

In a particular aspect, an effective amount of a neutralizing agent, for example an antibody, is an amount that inhibits Nrp-1 or PD-1, typically that inhibits or neutralizes Nrp-1 or PD-1 inhibitory activity towards anti-tumor immune response.

Typically, the effective amount of the neutralizing agent, for example of the antibody or fragment thereof, to be administrated to a patient may range from about 0.01 mg/kg to 500 mg/kg of body weight for a human patient. In a particular embodiment, each of the neutralizing agents, for example each of the antibodies or fragments thereof, is administered, typically through IV administration, at a dose of about 0.1 mg/kg to 100 mg/kg of a subject's body weight, for instance from 0.5 mg/kg to 10 mg/kg of a subject's body weight, for example 2, 3, 4, 5, 6, 7, 8 or 9 mg/kg of a subject's body weight, which may be adapted depending on the route of administration.

The present disclosure is further directed to a method of killing cancer cells in vitro, ex vivo or in vivo and to a method of treating cancer in a subject, these methods comprising exposing/contacting cancer cells to/with, or administering the subject suffering of a cancer with, an effective amount of a neuropilin-1 (Nrp-1) neutralizing agent and an effective amount of a programmed cell death-1 (PD-1) neutralizing agent and/or with an effective amount of a population of Nrp-1⁺ CD8⁺ tumor-infiltrating T lymphocytes (TILs) exhibiting anti-cancer cell effector functions and/or enhanced cancerous tumor infiltration ability as herein described, subsequently (the anti-Nrp-1 and anti-PD-1 neutralizing combination being administered first) or concomitantly.

As used herein, “treatment” or “treat” refers to therapeutic intervention in an attempt to alter the natural course of the subject being treated, and is typically performed for curative purpose. Desirable effects of treatment include, but are not limited to, preventing recurrence of disease, alleviation of symptoms, and diminishment of any direct or indirect pathological consequences of the disease, decreasing the rate of disease progression, amelioration or palliation of the disease state, and remission or improved prognosis. Typically, the treatment will induce an efficient therapeutic response of the immune system of the subject, typically efficient CD4⁺ and/or CD8⁺ T cells response(s).

By inducing a T cell response is typically meant herein that a T cell response directed towards one or several specific antigen is elicited. Before said induction, said T cell response was not present, or below detection levels or not functional. By enhancing a T cell response is meant herein that the overall action of T cells directed towards a certain antigen is made higher and/or more efficient compared to the overall action of said T cells before said enhancement. For instance, after said enhancement more T cells directed towards said antigen may be generated. As a result, the action of the additionally generated T cells increases the overall action against said antigen. Alternatively, said enhancement may comprise the increment of the action of T cells directed towards said antigen. Typically, T cells exhibiting an exhausted state before exposition to the combination of the invention, in particular CD8⁺ T cells, become activated after exposition to said combination. As a result, the action of the activated T cells increases the overall action against said antigen. Said T cells may for instance more efficiently infiltrate tumor cells and may react stronger and/or quicker with said antigen. Of course, the result of said enhancement may be generation of additional T cells together with increment of the action of said T cells.

Alternatively, said enhancement may comprise generation of additional T cells, increase of tumor infiltration, or increment of the action of T cells, only.

In preferred embodiments, compositions and methods of the invention are used to delay development of a cancer or to slow the progression of a cancer, typically of tumor growth.

The present disclosure is also directed to the use of a Nrp-1 neutralizing agent and of a PD-1 neutralizing agent, and/or of TILs, in particular Nrp-1⁺ CD8⁺ TILs, which have been “activated” after having been exposed to such Nrp-1 and PD-1 neutralizing agents (also herein identified as “activated TILs”), for the manufacture/preparation of a composition, preferably of a medicament, for killing cancer cells or for treating cancer.

Also herein disclosed is a composition, typically a pharmaceutical composition, preferably a medicament, comprising a Nrp-1 neutralizing agent and/or a PD-1 neutralizing agent, preferably the combination of a Nrp-1 neutralizing agent and of a PD-1 neutralizing agent, advantageously together with activated TILs, and a pharmaceutically acceptable carrier.

As already explained, neutralizing agents and any other active product present in the composition such as activated TILs, are typically in therapeutically effective amounts.

The pharmaceutically acceptable carrier is typically a non-toxic and sterile carrier such as physiological saline, non-toxic buffers, preservatives and the like. In a specific aspect, the term “pharmaceutically acceptable” means approved by a generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the neutralizing agents of the combination of the invention, preferably in purified form, together with a suitable amount of carrier so as to provide the form for proper administration to the subject. The formulation should suit the mode of administration. In a preferred aspect, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

Generally, the ingredients (neutralizing agents) of the combination and activated TILs, when present, are supplied either separately or mixed together in unit dosage form, for example, as a dry lyophilized powder or water free concentrate in a hermetically sealed container such as an ampoule or sachet indicating the quantity of active agent(s). Where the combination or composition is to be administered by infusion, it can be dispensed with an infusion bottle containing sterile pharmaceutical grade water or saline. Where the combination or composition is administered by injection, an ampoule of sterile water for injection or saline can be provided so that the ingredients may be mixed prior to administration.

In a particular aspect, the compounds of interest of the invention (typically the antibodies, or antigen-binding fragments, variants, or derivatives thereof), or composition comprising said compounds, can be administered to the subject by oral route, parenteral route, inhalation or topical route. The term parenteral as used herein includes, e.g., intravenous (IV), intraarterial (IA), intratumoral (IT), intraperitoneal (IP), intramuscular (IM), subcutaneous, rectal or vaginal administration.

While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc.

In particular methods compatible with the teachings herein, the combination of the invention, including for example antibodies or antigen-binding fragments, variants, or derivatives thereof can be delivered directly to the site of the adverse cellular population, typically intratumorally (IT), thereby increasing the exposure of the diseased tissue to the therapeutic agent.

In a particular aspect, Nrp-1⁺ TILs are isolated using anti-Nrp-1 mAb, amplified ex vivo in the presence of recombinant IL-2, and then reinjected to patients as described by Rosenberg SA's team [Chandran S S et al, Lancet Oncol. 2017 June; 18(6):792-802. Parkhurst M, Clin Cancer Res. 2017 May 15; 23(10):2491-2505]. Patients are in a particular aspect treated with lymphodepleting conditioning chemotherapy (such as a combination of cyclophosphamide and fludarabine), followed by a single intravenous infusion of autologous Nrp-1⁺ TILs in combination with IL-2 as described in the art (cf. Chandran S S et al., Lancet Oncol. 2017 June; 18(6):792-802; Mehta G U et al., J. Immunother. 2018 June; 41(5):241-247; and Lu Y C et al. J Clin Oncol. 2017 Oct. 10; 35(29):3322-3329).

Any compound of the composition of the invention may be administered to the subject daily (one time a day) during several consecutive days, for example during 2 to 10 consecutive days, preferably from 3 to 6 consecutive days. Said treatment may be repeated during 1, 2, 3, 4, 5, 6 or 7 weeks, or every two or three weeks or every one, two or three months. Alternatively, several treatment cycles can be performed, optionally with a break period between two treatment cycles, for instance of 1, 2, 3, 4 or 5 weeks.

Anyone of the herein described compounds of the invention can for example be administered as a single dose once a week, once every two weeks, or once a month. The treatment may be repeated one or several times per days, week, month or year.

Doses are administered at appropriate intervals which can be determined by the skilled person. The effective amount chosen will depend on multiple factors, including the route of administration, duration of administration, time of administration, the elimination rate of the compounds of the combination of the invention, or of the additional therapeutic compound(s) optionally used in combination with the compounds of said combination, the age, weight and physical condition of the patient and his/her medical history, and any other information known in medicine.

In a particular aspect, the compounds of the combination, or composition, can be delivered in a controlled release system known by the skilled person in the art.

Neutralizing agents of the combination are, as indicated previously, for separate (for example any neutralizing agent prior to or after the other neutralizing agent), concurrent or sequential use in a subject in need thereof. Concurrent/simultaneous administration of the neutralizing agents is preferred, in particular via IV route.

In a particular aspect, alternate administration of each neutralizing agent is possible, in particular via IV route.

In another particular aspect, the anti-Nrp-1 neutralizing agent and anti-PD-1 neutralizing agent may be administered simultaneously via different routes, the anti-Nrp-1 neutralizing agent being preferably administered through IT route and the anti-PD-1 neutralizing agent being administered through IV route.

In a particular aspect, these methods further comprise a step of exposing/contacting cancer cells to/with, or administering the subject suffering of a cancer with, an additional therapeutic agent known by the skilled person for treating cancer.

In another particular aspect, the treatment involving the use of a Nrp-1 neutralizing agent and of a PD-1 neutralizing agent, and optionally and advantageously activated TILs, for the manufacture/preparation of a composition for killing cancer cells or for treating cancer, is in further combination with an additional therapeutic agent known by the skilled person for treating cancer.

Such an additional therapeutic agent is for example a chemotherapeutic agent, an immune checkpoint blocker (“ICB”), an anti-cancer vaccine (“cancer vaccine”) or CAR-T cells.

The additional therapeutic agent may be administered separately, for example prior to or after, or concurrently with anyone of, or both, herein described neutralizing agents.

In another particular aspect, these methods further comprise a step of exposing cancer cells or the subject suffering of a cancer, to radiations, typically to radiotherapy. The radiotherapy typically involves rays selected from X-rays (“XR”), gamma rays and/or UVC rays.

The treatment which can include several anticancer agents is selected by the cancerologist depending on the specific cancer to be prevented or treated, and/or of the selected route.

The methods and combination of neutralizing agents of the invention, optionally together with any additional therapeutic agent, can be tested in vitro, and then in vivo for the desired therapeutic activity, prior to use in humans. For example, in vitro assays to demonstrate the therapeutic utility of a compound or pharmaceutical composition include the effect of a compound on a cell line or a patient tissue sample.

The effect of the compound or composition on the cell line and/or tissue sample can be determined utilizing techniques known to those of skill in the art including, but not limited to, cell proliferation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Another object herein described relates to a method of increasing, or of producing an efficient immune response, in a subject, typically against a specific target, preferably tumor antigen(s) or cancer/tumor cell or tissue, the method comprising administering, for example injecting, to said subject a combination according to the invention comprising a neuropilin-1 (Nrp-1) neutralizing agent and a programmed cell death-1 (PD-1) neutralizing agent or composition according to the invention comprising such a combination of compounds and preferably activated TILs, typically in an effective amount.

The detection of a therapeutically efficient immune response, preferably leading to the treatment of cancer, can be easily determined by the skilled person thanks to an in vitro or in vivo assay, for example thanks to technologies such as enzyme-linked immunosorbent assay (ELISA) in particular enzyme-linked immunospot (ELISPOT), immunohistochemistry (IHC), fluorescence in situ hybridization (FISH), radioimmunoassay (RIA), polymerase chain reaction (PCR), delayed type hypersensitivity response, intracellular cytokine staining, and/or extracellular cytokine staining, for example CD107a T-cell surface staining, and/or ⁵¹Cr chromium release assays.

The present disclosure is in particular directed to a combination comprising an effective amount of a Nrp-1 neutralizing agent and an effective amount of a PD-1 neutralizing agent, for use in activating PD-1⁺ CD8⁺ tumor-infiltrating T lymphocytes (TILs) anti-cancer cell effector functions and/or for enhancing cancerous tumor infiltration by PD-1⁺ CD8⁺ TILs having active anti-cancer cells effector functions.

In a particular aspect, the present combination of the invention allows T-lymphocytes, in particular CD8⁺ T cells, preferably Nrp-1⁺ and/or PD-1⁺ CD8⁺ T cells, displaying an exhausted state (also herein identified as “dysfunctional T lymphocytes” or “dysfunctional TILs”) to be restored into their effector functions towards tumor cells, i.e. to be able to infiltrate/invade tumors, to proliferate, and kill tumor cells, decrease tumor size and/or control tumor growth and/or progression or tumor cell proliferation. Thus, the anti-Nrp-1 neutralising agent, in particular when associated with an anti-PD-1 neutralising agent, advantageously re-establishes the functionality of these T cells.

Typical examples of CD8⁺ T cells displaying an exhausted state, as well as typical examples of CD8⁺ T cells advantageously capable of recognizing tumor cells, are typically CD8⁺ T cells coexpressing Nrp-1 and PD-1, preferably high levels of PD-1 such as PD-1^{T} lymphocytes (Thommen et al.³⁵). Thus, said CD8⁺ T cells displaying an exhausted state/capable of recognizing tumor cells may be identified by determining the expression levels of Nrp-1 and of PD-1, preferably of high levels of PD-1, by CD8⁺ T cells, and preferably also, the presence of anyone, at least two or all of the following receptors: CTLA-4, Tim-3, LAG-3, VISTA, BTLA, TIGIT, 2B4 (CD244) and KLRG1, preferably two or all of the following receptors: CTLA-4, Tim-3 and LAG-3.

Thus, also herein described is a method, for example an in vitro or ex vivo method, for detecting CD8⁺ TILs capable of recognizing tumor cells, in particular PD-1^{T} TILs, the method comprising determining i) whether said TILs express Nrp-1, and optionally ii) whether said TILs express one or several receptors selected from CTLA-4, Tim-3, LAG-3, VISTA, BTLA, TIGIT, 2B4 (CD244) and KLRG1, preferably from CTLA-4, Tim-3 and LAG-3, and/or iii) the PD-1⁺ status of said TILs, the expression of Nrp-1 by the TILs indicating that said TILs are capable of recognizing tumor cells, typically that said TILs display high tumor recognition capacity.

In a particular aspect, the presence of Nrp-1⁺ PD-1⁺ CD8⁺ TILs is indicative that said TILs are dysfunctional.

In another particular aspect, the presence of Nrp-1⁺ PD-1⁺ CD8⁺ TILs further expressing one or several receptors among those identifies herein above, for example selected from CTLA-4, Tim-3 and LAG-3, is indicative that said TILs are dysfunctional.

In a particular aspect, the herein above described method for detecting CD8⁺ TILs capable of recognizing tumor cells is performed in vitro or ex vivo and comprises a first step of providing a biological sample comprising TILs.

In the context of the present invention, the biological sample is selected from tumor tissue (a tumor tissue “specimen” or “biopsy”), a sample of cancer tissue. The biological sample is typically a tumor sample, in particular the tumor sample of a subject as herein described (including tumor cells and TILs).

The detection may be performed thanks to antibodies capable of respectively detecting Nrp-1 and PD-1, preferably specifically or preferentially, typically of recognizing and binding Nrp-1 and PD-1, as well as any fragment, variant, derivative or analogue thereof as herein defined and that are not required to neutralize Nrp-1 and/or PD-1, and optionally thanks to additional antibodies capable of recognizing and binding CTLA-4, Tim-3, LAG-3, VISTA, BTLA, TIGIT, 2B4 (CD244) and/or KLRG1, preferably CTLA-4, Tim-3 and/or LAG-3, as well as any fragment, variant, derivative or analogue thereof as herein defined, and that are not required to exhibit a neutralizing action towards CTLA-4, Tim-3, LAG-3, VISTA, BTLA, TIGIT, 2B4 (CD244) and/or KLRG1, preferably towards CTLA-4, Tim-3 and/or LAG-3, or their respective ligands.

The determination that Nrp-1 and PD-1 and optionally one or several receptors among those identified herein above, for example CTLA-4, Tim-3 and/or LAG-3, are expressed for example on the surface of a significant proportion of TILs, typically on the surface of at least 5%, 6%, 7%, 8%, 9%, 10%, 15%, 20%, 25%, 30%, 35%, 40%, 45%, 50%, 55%, 60%, 65%, 70%, 75%, 80%, 85% or 90% of TILs, in particular up to 40% for Nrp-1 and up to 90% for PD-1 of NSCLC TILs, from the subject, being the indication that CD8⁺ T cells display an exhausted state (are dysfunctional)/are capable of advantageously recognizing tumor cells.

The PD-1 status (i.e. level of PD-1 at the surface) of T cells may be determined for example by surface expression determined by immunofluorescence analysis (cf. Thommen et al.³⁵). Experiments performed provide an example of such analysis. In these experiments, resected solid tumor lesions were processed into single-cell suspensions by mechanical dissociation and enzymatic digestion using accutase (PAA), collagenase IV (Worthington), hyaluronidase (Sigma) and DNAse type IV (Sigma). All samples were cryopreserved until further usage. For phenotypic characterization of cryopreserved tumor digests, samples were thawed, washed, resuspended in 50 μl PBS and blocked with Fc receptor blocking agent (eBioscience) for 20 min at 4° C. Cells were stained with live/dead Zombie UV (Invitrogen). Cells were washed, resuspended in 50 μl of staining buffer (PBS, 2 mM EDTA, 0.1% NaN₃, 2% FCS) containing antibodies for surface staining and incubated for 20 min at 4° C. Corresponding isotype antibodies were used as a control. After washing twice, secondary antibodies were added where indicated for 20 min at 4° C. After washing, cells were taken up in 200 μl Fixation Buffer (eBioscience). Antibodies to PD-1 PE-Cy7 (EH12.1), PD-1 AlexaFluor647 (EH12.1) were purchased from BD.

The T cell effector functions may be confirmed by detection and/or measure for example of the tumor infiltration by CD8⁺ T cells, preferably by CD8⁺ T cells displaying high tumor recognition capacity as herein referred to, by detection and/or measure of the externalization of a cytotoxic marker, such as CD107a, at the T cell surface, and/or by detection and/or measure of TCR-mediated cytotoxicity of the T cell toward the tumor by chromium release assay.

The detection and/or measure of the tumor infiltration by CD8⁺ T cells may be performed for example by immunohistochemistry (THC), using for example anti-CD3 and/or anti-CD8 mAb on formalin-fixed paraffin-embedded (FFPE) tumors, or by immunofluorescence analysis of a freshly resected and dissociated tumor (as described above, Thommen et al.³⁵) using anti-CD3 and/or anti-CD8 mAb.

Increase of tumor infiltration by CD8⁺ T cells is typically associated to an increase of the CD8⁺ T-cell/CD4⁺ T-cell ratio in the tumor.

The detection and/or measure of the externalization of a cytotoxic marker, such as CD107a, at the T cell surface may be performed for example by cell surface immunofluorescence analysis (FACS).

The detection and/or measure of TCR-mediated cytotoxicity of the T cell toward the tumor may be performed for example by CD107a externalisation, chromium release assay or long-term assays (xCELLigence).

The T cell effector functions may also be confirmed by detection and/or measure of the restoration of the migratory capacity of CD8⁺ T cells, typically of PD-1⁺ CD8⁺ T cells, preferably of Nrp-1⁺ PD-1+CD8⁺ T cells, in particular Nrp-1⁺ PD-1^{hi} T cells, for example by in vitro transwell assay.

A particular aspect of the present disclosure relates to a method, for example an in vitro or ex vivo method, for predicting the response of a subject to anti-PD-1 treatment of cancer, the method comprising detecting CD8⁺ TILs recognizing cancer cells as herein described. This method is preferably applied to a subject who has not been previously exposed to a Nrp-1 neutralizing agent.

The presence in (a biological sample of) the subject of TILs expressing Nrp-1, in particular of Nrp-1⁺ PD-1⁺ CD8⁺ TILs, for example of T cells expressing Nrp-1 and PD-1 as well as CTLA-4, Tim-3 and/or LAG-3, is indicative that the subject does not (typically might) respond, or does not (typically might) optimally respond, to the anti-PD-1 treatment of cancer. On the contrary, the absence in (a biological sample of) the subject of TILs expressing Nrp-1, in particular of Nrp-1⁺ PD-1⁺ CD8⁺ TILs, for example of T cells expressing Nrp-1 and PD-1 as well as CTLA-4, Tim-3 and/or LAG-3, is indicative that the subject does not (typically might) respond to the anti-PD-1 treatment of cancer.

Is also herein described a method, for example an in vitro or ex vivo method, for identifying a subject who respond therapeutically to a treatment of cancer with an antibody combination therapy comprising an effective amount of a Nrp-1 neutralizing antibody and an effective amount of a PD-1 neutralizing antibody, the method comprising detecting CD8⁺ TILs recognizing cancer cells as herein described, the presence in (a biological sample of) the subject of TILs expressing Nrp-1, in particular of Nrp-1⁺ PD-1⁺ CD8⁺ TILs, being indicative that the subject responds (typically might respond) to the antibody combination therapy, and the absence in (a biological sample of) the subject of TILs expressing Nrp-1, in particular of Nrp-1⁺ PD-1⁺ CD8⁺ TILs, being indicative that the subject does not respond (typically might not respond), or does not (typically might not) optimally respond, to the antibody combination therapy.

In a particular aspect, is also herein described an in vitro or ex vivo method for predicting the response of a human subject to a treatment of lung cancer combining, in a particular aspect, anti-PD-1 and anti-Nrp-1 agents, the method comprising detecting CD8⁺ TILs recognizing cancer cells (as herein described), the presence, in the TILs of a biological sample of the subject, of about 15±5% of Nrp-1⁺ PD-1⁺ CD8⁺ TILs being indicative that the human subject respond to the treatment of cancer combining anti-PD-1 and anti-Nrp-1 agents, and the presence, in the TILs of a biological sample of the subject, of a different relative percentage of Nrp-1⁺ PD-1⁺ CD8⁺ TILs, typically a smaller percentage, being indicative that the human subject does not respond, or does not optimally respond, to the treatment of cancer combining anti-PD-1 and anti-Nrp-1 agents. For non-responding subjects, the administration of a composition as herein described comprising activated TILs and any additional active compound of interest as herein described is a therapeutic option allowing the treatment of the cancer the subject is suffering of.

Also herein described is a method of designing a cancer treatment for a subject in need thereof comprising testing for the presence of CD8⁺ T cells displaying an exhausted state as herein described in (a biological sample from) the subject.

In a particular aspect, inventors herein describe a method for treating cancer in a subject, wherein the method comprises a step of administering to a subject having a cancer (as herein described) Nrp-1⁺ CD8⁺ TILs exhibiting anti-cancer cell effector functions and/or enhanced cancerous tumor infiltration ability, typically a population thereof, wherein said TILs have been obtained from the subject having a cancer and have been preferably amplified ex vivo before the administration step. In a preferred aspect Nrp-1⁺ CD8⁺ TILs are PD-1^{high} CD8⁺ TILs, in particular PD-1^{T} TILs.

In another particular aspect, inventors herein describe a method for treating cancer in a subject, wherein the method comprises a step of administering an antibody combination comprising an effective amount of i) a Nrp-1 neutralizing antibody and ii) an immunotherapeutic antibody to a subject having a cancer (as herein described), said subject expressing Nrp-1⁺ CD8⁺ TILs, in particular Nrp-1⁺ PD-1⁺ CD8⁺ TILs. The immunotherapeutic antibody is any antibody well-known by the skilled person to be immunotherapeutic. Such an immunotherapeutic antibody may be selected for example from a PD-1 neutralizing antibody, a TIM-3 neutralizing antibody, a LAG-3 neutralizing antibody and a CTLA-4 neutralizing antibody. In a preferred aspect, the immunotherapeutic antibody is a PD-1 neutralizing antibody.

In another particular aspect, the method further comprises a step iii) of administering to the subject additional Nrp-1⁺ CD8⁺ TILs exhibiting anti-cancer cell effector functions and/or enhanced cancerous tumor infiltration ability (typically activated CD3⁺ CD8⁺ TILs), preferably additional Nrp-1⁺ CD8⁺ TILs which are autologous to the subject and which have been amplified ex vivo before said administration step.

A human Nrp-1 antibody capable of recognizing (i.e. of binding) human Nr-p-1, without necessary neutralizing it (contrary to other antibodies herein above described), can be selected from the group consisting of any known human Nrp-1 binding antibodies, for example from the group consisting of AF3870 (R&D system), 12C2 (Biolegend), and a variant or a derivative thereof that retain the binding specificity for human Nrp-1.

A mouse Nrp-1 antibody capable of recognizing (i.e. of binding) mouse Nr-p-1, without necessary neutralizing it (contrary to other antibodies herein above described), can be selected from the group consisting of 3E12 and a variant or a derivative thereof that retain the binding specificity for mouse Nrp-1.

A human PD-1 antibody capable of recognizing (i.e. of binding) human PD-1, without necessarily neutralizing it (contrary to other antibodies herein above described), can be selected from the group consisting of any known human PD-1 neutralizing antibodies, for example from the group consisting of J105 (eBioscience), and a variant or a derivative thereof that retain the binding specificity for human PD-1.

The present disclosure is also directed to a kit comprising at least two neutralizing agents selected from a Nrp-1 neutralizing agent (for example an anti-Nrp-1 antibody), a PD-1 neutralizing agent (for example an anti-PD-1 antibody), and anyone of the herein described immunotherapeutic agent or composition, preferably purified neutralizing agents, preferably in different containers/packages, and optionally an additional therapeutic agent as herein described.

Neutralizing agents may also be in the same container. The container(s) may be hermetically sealed container(s) such as for example ampoule(s), sachet(s), syringe(s) or infusion bottle(s).

Another kit herein described comprises i) a population of Nrp-1⁺ CD8⁺ tumor-infiltrating T lymphocytes (TILs) exhibiting anti-cancer cell effector functions and/or enhanced cancerous tumor infiltration ability, preferably PD-1^{high} TILs, in particular PD-1^{T} TILs, even more preferably TILs which have been obtained from the subject to be treated and have been amplified ex vivo, and ii) at least one immunotherapeutic agent, preferably an immunotherapeutic antibody, the immunotherapeutic agent being preferably a purified immunotherapeutic agent. The kit may also comprise an additional therapeutic agent as herein described (this additional therapeutic agent being different from said population of TILs and from said at least one immunotherapeutic agent).

The immunotherapeutic agent may be selected for example from a Nrp-1 neutralizing agent (for example anti-Nrp-1 antibody), a PD-1 neutralizing agent (for example anti-PD-1 antibody), a TIM-3 neutralizing agent (for example a TIM-3 neutralizing antibody), a LAG-3 neutralizing agent (for example a LAG-3 neutralizing antibody) and a CTLA-4 neutralizing agent (for example a CTLA-4 neutralizing antibody), and is preferably selected from a PD-1 neutralizing antibody, a TIM-3 neutralizing antibody, a LAG-3 neutralizing antibody and a CTLA-4 neutralizing antibody, or from a TIM-3 neutralizing antibody, a LAG-3 neutralizing antibody and a CTLA-4 neutralizing antibody.

A particular kit comprises multiple containers/packages of the single-dose combination or pharmaceutical composition each containing an effective amount of one or several of the herein above described neutralizing agents, for example of the Nrp-1 and/or PD-1 neutralizing agents, for a single administration in accordance with the herein described methods.

Optionally associated with such container(s) can be a notice providing instructions for using the neutralizing agents in any of the methods herein described, e.g. comprising administration schedules, to allow the correct (therapeutically efficient) administration of the products or composition contained therein to a subject in need thereof.

Instruments or devices necessary for administering the pharmaceutical composition(s) also may be included in the kits. The kit can for example include syringe(s), for example empty or pre-filled syringes. In a particular aspect, the kit is a kit for use for treating cancer in a subject.

Further aspects and advantages of the present invention will be disclosed in the following experimental section and figures which shall be considered as illustrative only.

### LEGENDS TO THE FIGURES

FIG. 1. Expression of Nrp-1 and PD-1 on T cells infiltrating human NSCLC tumors.

a. Surface expression of Nrp-1 on CD4⁺ and CD8⁺ T cells from TIL and PBL. TIL from freshly resected NSCLC tumors were isolated and then directly analysed by flow cytometry for Nrp-1 expression on CD3⁺ CD4⁺ and CD3⁺ CD8⁺ lymphocytes. Expression of Nrp-1 on CD4⁺ and CD8⁺ T cells from HD and NSCLC patient's PBL was evaluated. Percentages of positive cells are indicated. Right: percentages of Nrp-1⁺ CD8⁺ and Nrp-1⁺ CD4⁺ T cells in TIL (n=28) and PBL from HD (n=12) and NSCLC patients (n=11). b. Expression of CD25 and PD-1 on Nrp-1⁺ CD8⁺ T cells from NSCLC TIL. Percentages of Nrp-1⁺ T cells among CD8⁺ TIL expressing or not CD25 or PD-1 (n=13-to-16). Right: dot plot showing co-expression of Nrp-1 and PD-1 on CD8⁺ TIL from one representative patient. c. Expression of CD25 and PD-1 on Nrp-1⁺ CD4⁺ T cells from NSCLC TIL. Percentages of Nrp-1⁺ T cells among CD4⁺ TIL expressing or not CD25 or PD-1 (n=9-to-20). Right: dot plot showing co-expression of Nrp-1 and PD-1 on CD4⁺ TIL from one representative patient. d. Expression of Foxp3 in Nrp-1⁺ CD4⁺ T cells. Percentages of Nrp-1⁺ T cells among CD4⁺ TIL expressing or not Foxp3. Right: dot plot for co-expression of Nrp-1 and PD-1 on CD4⁺ TIL expressing or not Foxp3 from one representative patient. Data presented as mean±SEM. * p<0.05; ** p<0.01 and *** p<0.001.

FIG. 2. Expression of Sema-3A and Nrp-1 in human lung tumor cell lines and CTL clone.

a. Sema-3A protein expression in human lung tumor cell lines. Total protein extracts from lung tumor cell lines were analysed by western blot using anti-Sema-3A mAb. The bronchial epithelial cell line 16HBE was included as a control. Full length and proteolytically processed proteins are indicated. Anti-F-actin was included as a loading control. b. Expression of Nrp-1 on the P62 CTL clone unstimulated and stimulated with anti-CD3 mAb. Right: Co-expression of Nrp-1 and CD25, and Nrp-1 and PD-1 on P62 T cells stimulated with immobilized anti-CD3. c. Sema-3A-Fc binds to Nrp-1 on the P62 CTL clone surface. The P62 T-cell clone was unstimulated or stimulated with anti-CD3 for 48 h, pre-incubated for 30 min with Sema-3A-Fc (12 μg/mL), and then labelled with mouse anti-human IgG Fc fragment secondary mAb. d. Sema-3A-Fc inhibits CTL clone migration toward a CXCL12 gradient. The P62 T-cell clone was stimulated with anti-CD3 for 48 h, pre-incubated for 30 min with BSA or Sema-3A-Fec, and then seeded in the upper chambers of transwell plates and exposed to a gradient of CXCL12 chemokine loaded in the lower chambers. The number of T cells that had migrated into the lower chambers was determined. Results are represented as mean chemotaxis index ±SD of triplicate samples. e. Cytotoxic activity of the CTL clone toward autologous tumor cells. The P62 T-cell clone was stimulated with plastic-coated anti-CD3 for 48 h, left in media for 24 h to detach anti-CD3 mAb and then pre-incubated in medium or with Sema-3A-Fc. Cytotoxicity toward the cognate IGR-Pub tumor cell line was determined by a conventional 4 h ⁵¹Cr release assay at indicated E:T ratios. Data shown correspond to one of 3 independent experiments. * p<0.05; ** p<0.01.

FIG. 3. Expression of Sema-3B and Nrp-1 in B16F10 mouse melanoma model.

a. Expression of Sema-3B in B16F10 tumor cells. Total protein extracts from B16F10 tumor cells cultured in vitro or isolated ex vivo from tumor grafts were analysed by western blot using anti-Sema-3B mAb. Anti-F-actin was included as a loading control. b. Surface expression of Nrp-1 on CD4⁺ and CD8⁺ T cells infiltrating B16F10 melanoma engrafted in C57BL/6 mice. TIL from individual tumors were isolated at day 15 after tumor cell inoculation using CD45 beads, and then labelled with anti-Nrp-1, -CD3, -CD4 and -CD8 mAb. T lymphocytes from spleens and TdLN of tumor-bearing mice were analysed in parallel. Percentages of positive cells are included. Right: percentages of Nrp-1⁺ cells among CD8⁺ and CD4⁺ T cells in TIL (n=30) and splenocytes and TdLN (n=16-to-30). c. Expression of Nrp-1 and Foxp3 in CD4⁺ T cells. T lymphocytes from tumors, spleens and TdLN of B16F10 melanoma-bearing C57BL/6 mice were analysed at day 15 by flow cytometry. Right: Percentages of Nrp-1⁺ cells among Foxp3⁺ and Foxp3⁻ CD4⁺ T lymphocytes from B16F10 (n=11-to-20). d. Expression of CD44 and CD62L on Nrp-1⁺ and Nrp-1⁻ CD8⁺ T cells from B16F10 TIL. Right: Distribution of Nrp-1⁺ cells among CD8⁺ TIL subpopulations among naive, effector and memory T cells (n=10). Results are representative of 3-5 independent experiments. Data are presented as mean±SEM. *** p<0.001.

FIG. 4. Expression of T-cell activation/exhaustion markers on the CD8⁺ TIL surface.

a. Expression of Nrp-1, PD-1, LAG-3, CTLA-4 and Tim-3 on CD8⁺ T cells from B16F10 TIL isolated at day 15. Down: percentages of Nrp-1⁺ T cells among CD8⁺ T cells expressing or not PD-1, Tim-3, LAG-3 or CTLA-4 (n=16-to-24). Data are means±SEM. *** p<0.001. b. Expression of Nrp-1 on CD8⁺ T cells correlated with that of PD-1. c. Expression of Nrp-1 on CD8⁺ TIL is restricted to a PD-1^{hi} T-cell population. Expression of Nrp-1 and PD-1 on CD8⁺ TIL; identification of three T-cell subsets: Nrp-1⁺ PD-1^{hi} (31%), Nrp-1⁻ PD-1⁺ (40%) and Nrp-1⁻ PD-1⁻ (24%). Down: Expression of exhaustion/activation markers, functional proteins and transcription factors in Nrp-1⁺ PD-1^{hi}, Nrp-1⁻ PD-1⁺ and Nrp-1⁻ PD-1⁻ CD8⁺ T-cell subsets. Heat maps including percentages of cells positive for expression of PD-1, Nrp-1, Tim-3, CTLA-4, LAG-3, granzyme B (GrzB) and Ki-67, and gMFI for NFATc1, IRF-4, Helios, Blimp-1 and T-bet. d. gMFI of PD-1 expression on Nrp-1⁺ PD-1^{hi}, Nrp-1⁻ PD-1⁺ and Nrp-1⁻ PD-1⁻ TIL subsets. Results are representative of 3 to 5 independent experiments.

FIG. 5. The Nrp-1⁺ PD-1^{hi} TIL subset is enriched with activated antigen-specific CD8⁺ T lymphocytes.

a. Staining of CD8⁺ TIL with Trp2 and gp100 dextramers. C57BL/6 mice were engrafted with B16F10 melanoma cells and then vaccinated with Trp2 and gp100 antigenic peptides as described in Materials and Methods. On day 15, CD8⁺ T cells were isolated from TIL, labelled with anti-CD8, -Nrp-1 and -PD-1 mAb, and Trp-2 and gp100 dextramers, and then percentages of antigen-specific T cells were determined. Right: Percentages of Trp2 and gp100 dextramer-positive T cells among Nrp-1⁺ PD-1^{hi}, Nrp-1⁻ PD-1⁺ and Nrp-1⁻ PD-1⁻ TIL. b. Cytoplasmic expression of IFNγ and TNFα in Nrp-1⁺ PD-1^{hi}, Nrp-1-PD-1⁺ and Nrp-1⁻ PD-1⁻ CD8⁺ T cells. TIL were stimulated for 4 h with autologous tumor cells, stained with anti-CD8, -Nrp-1 and -PD-1 mAb and, after membrane permeabilization, with anti-IFNγ and -TNFα. mAb (n=18). c. Degranulation capacity of CD8⁺ TIL. TIL were stimulated with autologous tumor cells; then, Nrp-1⁺ PD-1^{hi}, Nrp-1⁻ PD-1⁺ and Nrp-1⁻ PD-1⁻ T-cell subsets were analysed for surface expression of CD107a. Mean percentages of CD107a⁺ lymphocytes are shown (n=16). d. Cytotoxic activity of freshly isolated CD8⁺ TIL toward B16F10 tumor cells. CD8⁺ TIL were pre-incubated in medium or with anti-Nrp-1, anti-PD-1, a combination of both blocking mAb or an isotype control; then, cytotoxicity toward autologous tumor cells was determined by 4 h and 12 h ⁵¹Cr release assay at 50:1 E:I ratio. e. Increase in MHC class I and PD-L1 expression on B16F10 tumor cells co-cultured with autologous CD8⁺ TIL. Kinetic studies of H-2K^{b}/D^{b} and PD-L1 expression on B16F10 cells co-cultured with CD8⁺ TIL for indicated time points. Expression profiles (left), percentages of positive cells (middle) and gMFI (right) of MHC-class I (upper panels) and PD-L1⁺ (lower panels) tumor cells are shown. f. Anti-Nrp-1 re-establishes migration of Nrp-1⁺ PD-1^{hi} T cells toward B16F10 tumor cell supernatant. TIL were pre-incubated for 30 min in medium or with neutralizing anti-Nrp-1, -PD-1, a combination of both mAb or an isotype control. Cells were seeded in the upper chambers of transwell plates and then exposed to a gradient of B16F10 supernatant loaded in the lower chambers. The numbers of Nrp-1⁺ PD-1^{hi}, Nrp-1⁻ PD-1⁺ and Nrp-1⁻ PD-1⁻ T cells that had migrated into the lower chambers were determined by flow cytometry. Results are representative of 3-to-4 independent experiments. Data presented as mean±SEM. * p<0.05; ** p<0.01 and *** p<0.001.

FIG. 6. Anti-Nrp-1 mAb combined with anti-PD-1 improve tumor progression control.

a. C57BL/6 mice were engrafted with B16F10 melanoma cells and then treated i.p. with anti-PD-1, i.t. with anti-Nrp-1, or a combination of both mAb injected in parallel at each site at days 6, 8, 10, 12 and 14 after tumor inoculation. An isotype control was included. Tumor volumes, measured every second day in mm³, are given as means (±SEM) of 5-to-7 mice/group. Mice were sacrificed at day 17, since tumor size exceeded the tolerated institutional limit. Data are means of two independent experiments out of three. * p<0.05; ** p<0.01 and *** p<0.001. b. Weight of tumors from mice untreated and treated with blocking mAb. Tumors were recovered at day 16 after engraftment and weighed. c. Absolute cell counts of CD3⁺ CD8⁺ TIL. Numbers of CD8⁺ T cells per milligram of tumor were determined at day 16 as described in Materials and Methods. d. Ratio of CD8⁺/CD4⁺ Treg cells in tumors from mice treated with blocking mAb or an isotype control. e. Absolute cell counts of KLRG1⁺, Ki-67⁺ and granzyme B+CD8⁺ T cells from B16F10 TIL. Numbers of T-cell subsets per milligram of tumor from mice treated with anti-Nrp-1, anti-PD-1, a combination of both mAb or an isotype control. Data are means from two independent experiments out of three. * p<0.05; ** p<0.01 and *** p<0.001.

FIG. 7. Expression of NRP, PLXN and SEMA3 genes in human lung tumor samples.

a. Relative expression of NRP1 and NRP2 transcripts in fresh NSCLC tumors performed by qRT-PCR analysis. b. Relative expression of PLXNA1, PLXNA2, PLXNA3, PLXNA4 and PLXND1 transcripts in NSCLC tumors performed by qRT-PCR analysis. c. Relative expression of SEMA3A, SEMA3B, SEMA3C, SEMA3D, SEMA3E, SEMA3F and SEMA3G transcripts in fresh human lung tumors. Expression was normalized to autologous healthy lung tissues (n=8).

FIG. 8. Expression of Nrp-1, Sema-3A and CXCR4 in human cells.

a. Expression of Nrp-1 on CD8⁺ T lymphocytes from HD PBL unstimulated (medium) and stimulated with immobilized anti-CD3 mAb. Right: Percentages of Nrp-1⁺ CD8⁺ T cells in HD PBL unstimulated and stimulated with anti-CD3 (n=10). b. Expression of Nrp-1 on CD4⁺ T lymphocytes from HD PBL unstimulated (medium) and stimulated with immobilized anti-CD3. Right: Percentages of Nrp-1⁺ CD4⁺ T cells in HD PBL unstimulated and stimulated with anti-CD3mAb (n=10). c. Expression of Sema-3A in human lung tumor cell lines. Intracellular immunofluorescence analysis of Sema-3A expression in NSCLC tumor cells. Full line: Anti-Sema-3A; dashed line: isotypic control mAb. d. Expression of CXCR4 chemokine receptor on the human P62 CTL clone surface. Full line: anti-CXCR4 mAb; dashed line: isotypic control.

FIG. 9. Expression of Sema-3B and Nrp-1 in the B16F10 mouse melanoma model.

a. Expression of Sema3 genes in B16F10 tumor cells cultured in vitro (left) or engrafted in C57BL/6 mice (right). Relative expression of Sema3A, Sema3B, Sema3C, Sema3D, Sema3E, Sema3F, 41 Sema3G and Sema4A transcripts in B16F10 tumor cells was determined by qRT-PCR analysis. b. Kinetic studies of Nrp-1 expression on CD8⁺ T cells from naive mouse splenocytes unstimulated or stimulated with immobilized anti-CD3 mAb. c. NFAT inhibitor inhibits Nrp-1 induction on CD8⁺ T cells. The NFAT inhibitor 11R-VIVIT inhibits expression of Nrp-1 induced by anti-CD3 activation of naive mouse splenocytes in a dose-dependent manner. d. Kinetic studies of Nrp-1, PD-1, LAG-3, CTLA-4 and Tim-3 induction on CD8⁺ TIL from B16F10 melanoma engrafted in C57BL/6 mice. Percentages of inhibitory receptor-expressing CD8⁺ T lymphocytes are shown at indicated time points. e. Absolute numbers of CD8⁺ TIL expressing Nrp-1, PD-1, LAG-3, CTLA-4 or Tim-3 per milligram of tumor. f. Expression of Batf; Cd244, Tigit and Egr2 genes in mouse CD8⁺ TIL. Relative expression of Batf; Cd244, Tigit and Egr2 transcripts in Nrp-1⁺ PD-1^{hi}, Nrp-1⁻ PD-1⁺ and Nrp-1⁻ PD-1⁻ T-cell subsets from B16F10 TIL was determined by qRT-PCR analysis at day 15 after tumor engraftment. g. Expression of inhibitory receptors on CD4⁺ TIL. Percentages of Nrp-1⁺ T cells co-expressing or not PD-1, Tim-3 or CTLA-4 among Foxp3⁻ CD4⁺ T lymphocytes from B16F10 melanoma (n=5-9). h. Expression of Ki-67 in Nrp-1⁺Foxp3⁻ CD4⁺ T cells. Percentages of Nrp-1⁺ T cells co-expressing or not Ki-67 among Foxp3⁻ CD4⁺ T lymphocytes infiltrating B16F10 melanoma (n=10).

FIG. 10. Tumor growth and infiltration of B16F10 melanoma.

a. C57BL/6 mice were engrafted with B16F10 cells and then vaccinated with Trp2 peptide delivered with poly(I:C) at indicated time points (arrows) or with poly(I:C) control alone (vehicle). Tumor volumes, measured every third day in mm³, are given as means (±SEM) of 5 mice/group. b. Kinetic studies of inhibitory receptor expression on CD8⁺ T cells infiltrating B16F10 tumors. Absolute numbers of CD8⁺ T cells expressing inhibitory receptors in TIL from Trp-2-vaccinated mice and control mice. Numbers of PD-1⁺, Nrp-1⁺, LAG-3⁺, CTLA-4⁺ and Tim-3⁺ CD8⁺ TIL per milligram of tumor are determined at indicated time points. c. H-2Kb/Db expression profiles of B16F10 cells cultured in vitro in medium alone or with IFNγ for 12 h, or isolated ex vivo at day 15. d. Growth of B16F10 tumors engrafted in C57BL/6 mice treated with blocking anti-Nrp-1, -PD-1, a combination of both mAb or an isotype control as described in Materials and Methods. Individual mouse tumors are shown.

FIG. 11. The Nrp-1⁺ PD-1^{hi} TIL subset is enriched with activated antigen-specific CD8⁺ T cells.

a. Cytotoxicity of freshly isolated CD8⁺ TIL. CD8⁺ TIL were pre-incubated in a medium or with anti-Nrp-1, anti-PD-1 or a combination of both mAb; then, cytotoxicity toward autologous tumour cells was determined.

b. Expression of perforin in CD8⁺ T cells. TILs were stimulated with autologous tumour cells in the absence or presence of neutralizing anti-Nrp-1, anti-PD-1 or anti-Nrp-1 plus anti-PD-1, then, T-cell subsets were analyzed for expression of perforin (n=5).

FIG. 12. Trp-2 peptide vaccine increases the number of Nrp-1+PD-1hi TILs that are enriched with activated antigen-specific CD8⁺ T cells.

a. Kinetic studies of inhibitory receptor expression on CD8⁺ T cells infiltrating B16F10. Absolute numbers of CD8⁺ T cells expressing inhibitory receptors in TILs from Trp-2-vaccinated mice and control mice. Numbers of PD-1⁺, Nrp-1⁺, LAG-3⁺, CTLA-4⁺ and Tim-3⁺ CD8⁺ TIL per milligram of tumour are determined.

b. Left: Staining of CD8⁺ TIL with dextramers. C57BL/6 mice were engrafted with B16F10 and then vaccinated with Trp2 and gp100 peptides. On day 15, TILs were isolated from tumours.

Right: Percentages of Trp2 (n=8) and gp100 (n=5) dextramer-positive T cells among Nrp-1⁺ PD-1^{hi} Nrp-1⁻ PD-1⁺ and Nrp-1⁻ PD-1⁻ CD8⁺ TIL.

FIG. 13. Nrp-1 is enriched on PD-1⁺ CD8⁺ TILs.

a. Expression of Nrp-1 in PD-1 CD8⁺ T cells subsets from NSCLC TILs. Percentages of Nrp-1 among CD8⁺ TILs expressing or not PD-1 (n=13).

b. Expression of Nrp-1 in PD-1 CD8⁺ T cells subsets from B16-F10, LLC, MC-38 and TC-1 mouse tumors.

### EXAMPLES

Abbreviations: CTL: cytotoxic T lymphocyte; CTLA-4: cytotoxic T lymphocyte antigen 4; MHC-I: major histocompatibility complex class I; mAb: monoclonal antibody; NSCLC: non-small-cell lung cancer; Nrp-1: neuropilin-1, PD-1: programmed cell death-1; qRT-PCR: quantitative real-time polymerase chain reaction; r: recombinant; Sema: semaphorin; TCR: T-cell receptor; TIL: tumor-infiltrating T lymphocyte; Treg: regulatory T.

Materials & Methods

Human Lung Tumors and Freshly Isolated Lung TIL

Fresh NSCLC (non-small-cell lung cancer) tumors were obtained from the Centre chirurgical Marie Lannelongue and the Institut mutualiste Montsouris. RNA was immediately extracted with TRIzol reagent (Invitrogen), reverse-transcribed and then subjected to qRT-PCR (quantitative real-time polymerase chain reaction).

For freshly isolated TIL (tumor-infiltrating T lymphocyte), human lung tumors were dissociated mechanically and enzymatically using a tumor dissociation kit (MACS, Miltenyi Biotec). Mononuclear cells were then isolated by a Ficoll-Hypaque gradient. All human experiments were approved by the Institutional Review Board of the Gustave Roussy Institute.

Derivation and Culture of the P62 CTL Clone and IGR-Pub Autologous Tumor Cell Line

NSCLC cell line IGR-Pub was derived from the tumor specimen of patient Pub adenocarcinoma as described¹⁷. Autologous CTL (cytotoxic T lymphocyte) clone P62 was derived from TIL of the same patient⁴⁹. T-cell clone P62 was stimulated every month with irradiated autologous IGR-Pub tumor cells and irradiated allogeneic Laz509 EBV-transformed B cells in RPMI-1640 medium supplemented with 10% human AB serum and rIL-2¹⁷.

The allogeneic NSCLC cell lines IGR-B2, IGR-Heu, ADC-Coco, ADC-Tor and ADC-Let were derived from tumor specimens in one of inventors' laboratories and maintained in culture as described⁴⁹. A549 (ADC), SK-Mes, Ludlu (SCC), DMS53 (SCLC), H460, H1155 (LCC) and H1355 (ADC) were previously reported⁵⁰.

The SV40-immortalized human bronchial epithelial cell line 16HBE14o- (16HBE), used as a control, was previously described⁵¹.

Quantitative Real-Time (RT)-PCR

Total RNA was immediately extracted from sorted cell populations using the Single Cell RNA Purification Kit (Norgen Biotek) or TRIzol reagent (Invitrogen) for human samples. cDNA was synthesised using the Maxima First Strand cDNA Synthesis Kit (ThermoFischer Scientific). qRT-PCR was performed on a Step-One Plus (Applied Biosystems) using Maxima SYBR Green Master Mix (ThermoFischer Scientific). Expression levels of transcripts were normalized to 18S expression. PCR primers and probes for human (NRP1-2, PLXNA1-4, PLXND1, SEMA3A-G, 18S) and mouse (Batf, Cd244, Tigit, Egr2, 18S) genes were designed by Sigma-Aldrich and used according to the manufacturer's recommendations.

B16F10 Melanoma Cell Line, Tumor Engraftment and Peptide Cancer Vaccine

The B16F10 melanoma cell line (H-2^{b}) was purchased from the American Type Culture Collection (ATCC). Tumor cells were grown in DMEM/F-12 medium (ThermoFischer Scientific) supplemented with 10% FCS, 2 mM L-glutamine, 1 mM sodium pyruvate, and antibiotics (50 U/ml penicillin and 50 g/ml streptomycin).

Female C57BL/6J mice were purchased from Envigo. For each experiment, groups of 4-to-10 mice 7-9 weeks of age received 2×10⁵ B16F10 melanoma cells subcutaneously (s.c.) in the right flank. All animals were housed at Gustave Roussy's animal facility and treated in accordance with institutional animal guidelines.

For the cancer vaccine, C57BL/6J mice were immunized s.c. with 100 μg of gp100 (KVPRNQDWL—SEQ ID NO: 8) and/or Trp2 (SVYDFFVWL—SEQ ID NO: 9) peptides (GeneCust) plus 25 μg of poly(I:C) adjuvant (InvivoGen), at day 5 and every week at the tail base.

Murine TIL Isolation

Tumors were harvested at days 8-to-20 and digested for 40 min at 37° C. according to Tumor Dissociation Kit protocol (Miltenyi Biotec). Tumors were crushed on 100 μm cell strainers and washed twice with PBS 2% fetal calf serum (FCS). Single cell suspensions were enriched for CD45⁺ cells or CD8⁺ cells using the MultiMACS system (Miltenyi Biotec). Briefly, cells from tumor tissues were labelled with anti-CD45 or anti-CD8a microbeads (Miltenyi Biotec), and then purified using the POSSEL program on MultiMACS. The positive fraction was recovered for TIL analysis by flow cytometry or ex vivo assays.

Antibodies and Flow Cytometry

For human cell surface and intracellular staining, anti-CD3 (UCHT1), -CD4 (RPA-T4), -CD8α (RPA-T8), -CD25 (CD25-3G10), -FoxP3 (259D), -PD-1 (J105) and -Nm-1 (12C2) mAb, and rSema-3A-Fc were used. Cell surface and intracellular staining of mouse cells was performed on single-cell suspensions using antibodies specific to the following molecules: CD3 (17A2), CD4 (RM4-5), CD8α (53-6.7), CTLA-4 (UC10-4B9), PD-1 (29F.1A12), Tim-3 (RMT3-23), LAG-3 (C9B7W), Nrp-1 (3E12), T-bet (REA102), NFATc1 (7A6), Blimp-1 (5E7), Helios (22F6), IRF-4 (REA201), Ki-67 (REA183) and granzyme B (GB11). Dead cells were excluded using the Live/Dead Fixable Blue Dead Cell Stain Kit (Invitrogen).

For intracellular staining, cells were fixed/permeabilized with the Foxp3 Staining Buffer Set according to the manufacturer's instructions (eBioscience). Staining of Trp2 and gp100-specific T cells was performed using H-2Kb/SVYDFFVWL and H-2Db/KVPRNQDWL dextramers, respectively (Immudex). Flow cytometric analysis was conducted on an LSR Fortessa (BD) and analyzed using FlowJo software (Tree Star).

In Vitro Migration Assay

Freshly isolated CD8⁺ TIL were incubated for 30 min to 1 h either in medium or in the presence of neutralizing anti-Nrp-1 mAb (R&D system MAB59941), anti-PD-1 (Biolegend clone RMP1-14) or isotype control (Biolegend Isotype Rat IgG₂a RTK2758). B16F10 tumor cells were cultured for 2 days in the lower chambers of Transwell plates (Corning) and then TIL were seeded in the upper chambers to trigger T-cell migration. After 2 h at 37° C., the number of CD8⁺ T cells that had migrated into the lower chambers was counted by flow cytometry and phenotyped.

For experiments with CTL clone P62, activated T cells were incubated for 30 min to 1 h either with BSA or Sema-3A-Fc (100 ng/ml, R&D system), and their ability to migrate toward the human rCXCL12 (50 nM) was evaluated. Results were expressed as chemotaxis index.

In Vitro T-Cell Stimulation, Cytokine Production and Cytotoxic Experiments

Purified CD45⁺ TIL were co-cultured for 4 h with B16F10 tumor cells, pulsed with Trp2 (1 μg) and gp100 (1 μg) peptides, in the presence of Brefeldin A (eBioscience), monensin (Merck) and anti-CD107a mAb (1D4B). TIL were stained with mAb specific for surface proteins prior to fixation and permeabilization. Permeabilized cells were then stained with anti-IFN-γ (XMG1.2) and anti-TNF-α (MP6-XT22) mAb.

For cytotoxicity experiments, freshly isolated CD8⁺ TIL were either kept in medium or pretreated with neutralizing anti-Nrp-1 (R&D system MAB59941), anti-PD-1 (clone RMP1-14) or isotype controls (Biolegend Isotype Rat IgG₂a RTK2758). Cytotoxic activity toward the B16F10 cell line, pulsed with Trp2 and gp100 peptides, was evaluated using a conventional 4 h overnight chromium (⁵¹Cr) release assay.

For experiments with CTL clone P62, activated T cells were incubated for 30 min to 1 h either with BSA or Sema-3A-Fc (100 ng/ml), and their cytotoxic activity toward the autologous tumor cell was evaluated.

Western Blot Analyses

Equivalent amounts of protein extracts from tumor cell lines were separated by SDS-PAGE and transferred to a nitrocellulose membrane as described⁵². Blots were then probed with rat anti-Sema-3B mAb (R&D system MAB5440), mouse anti-Sema-3A (R&D system MAB1250) or anti-β-actin-peroxidase (Merck A3854), followed by secondary HRP-conjugated Ab.

In Vivo PD-1 and Nrp-1 Blockade

Mice were treated i.p. with 100 μg/mouse of anti-PD-1 (Bio-X-Cell; RMP1-14) mAb and/or i.t. with 25 μg/mouse of anti-Nrp-1 (R&D system; MAB59941) mAb. For tumor outgrowth experiments, mice were treated on days 6, 8, 10, 12 and 14 after tumor inoculation, and TIL were sorted and analysed on day 16.

Statistical Analysis

Statistical significance was determined with the one-way or two-way ANOVA test with Bonferroni correction, or with the two-tailed Student t test (GraphPad Prism, GraphPad software).

Results

Nrp-1 is Expressed on a Subset of CD8⁺ T Lymphocytes Infiltrating Human Lung Tumors

Nrp-1 is expressed on CD4⁺ Treg cells in human lymph nodes and in TIL from colorectal cancer metastases.^{14,15} However, little is known about its expression on CD8⁺ TIL. Therefore, inventors first evaluated the expression of NRP1 transcripts in primary human lung tumors and autologous normal lungs. Quantitative real-time PCR (qRT-PCR) showed high expression levels of NRP1 mRNA in some lung tumor samples compared to the cognate normal lung (FIG. 7a). Moreover, freshly isolated lung TIL were found to express NRP1 mRNA (data not shown). Human NSCLC were also found to express NRP2 and certain Plexin (Plxn) A or D family member transcripts, as well as genes encoding Np ligands of the Sema 3 family (FIG. 7).

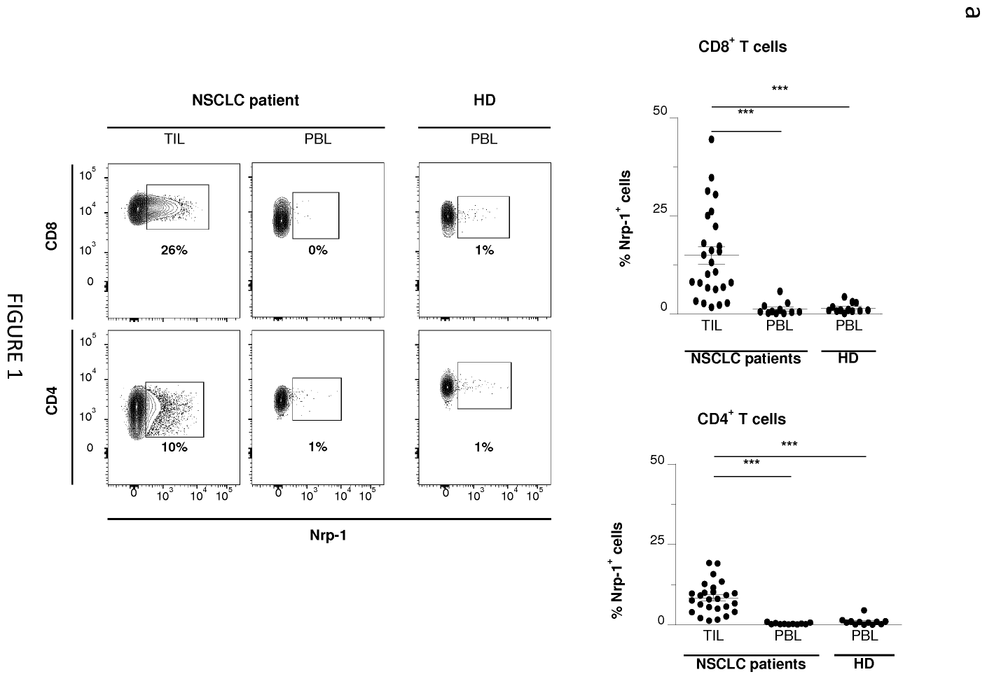

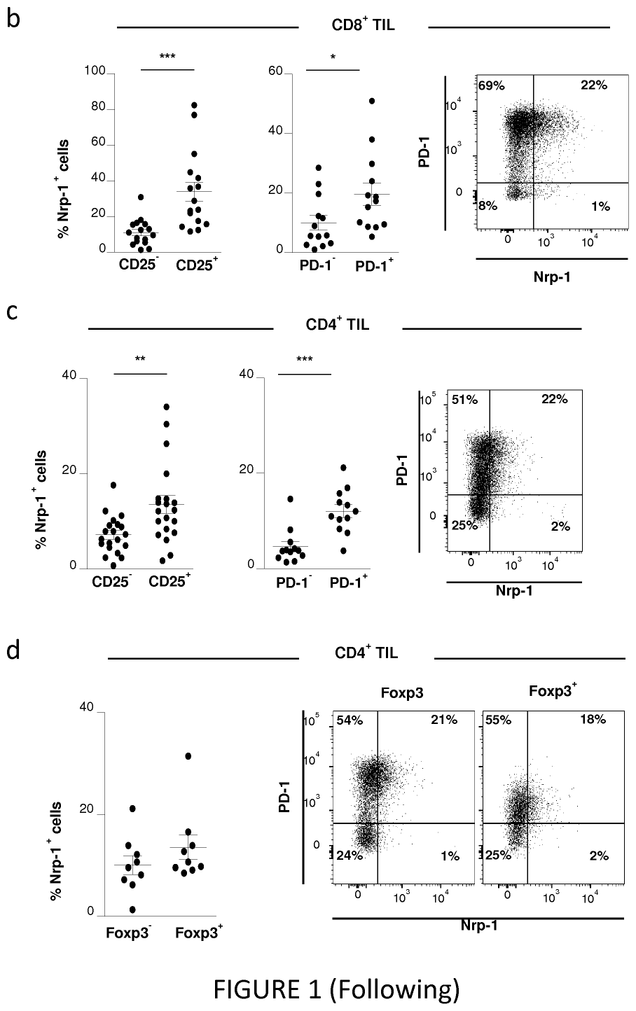

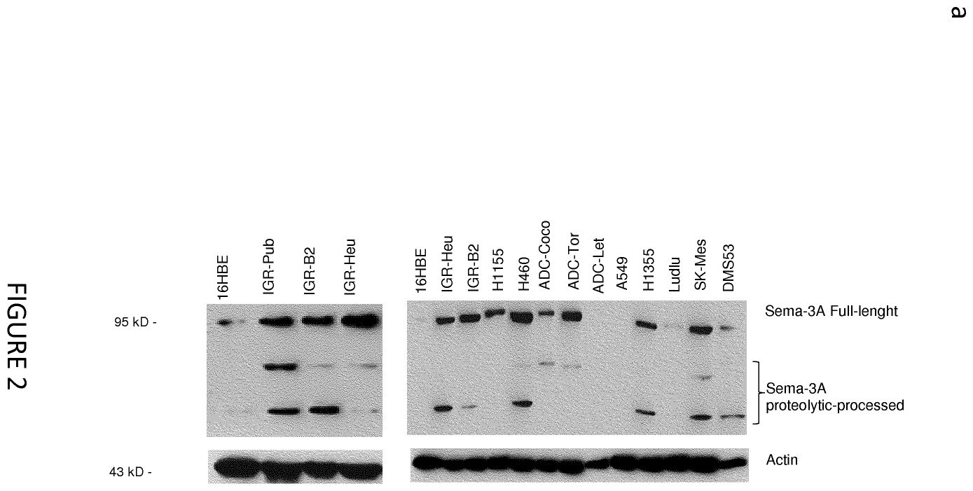

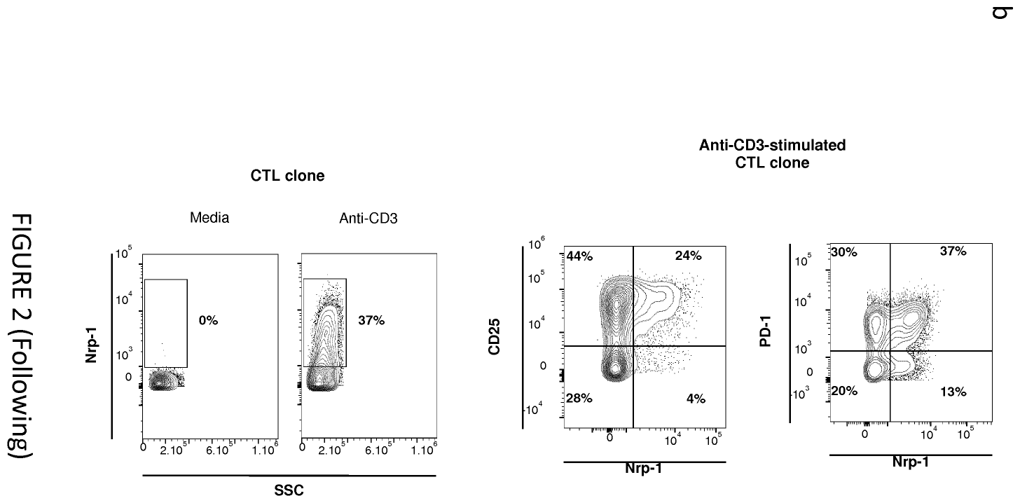

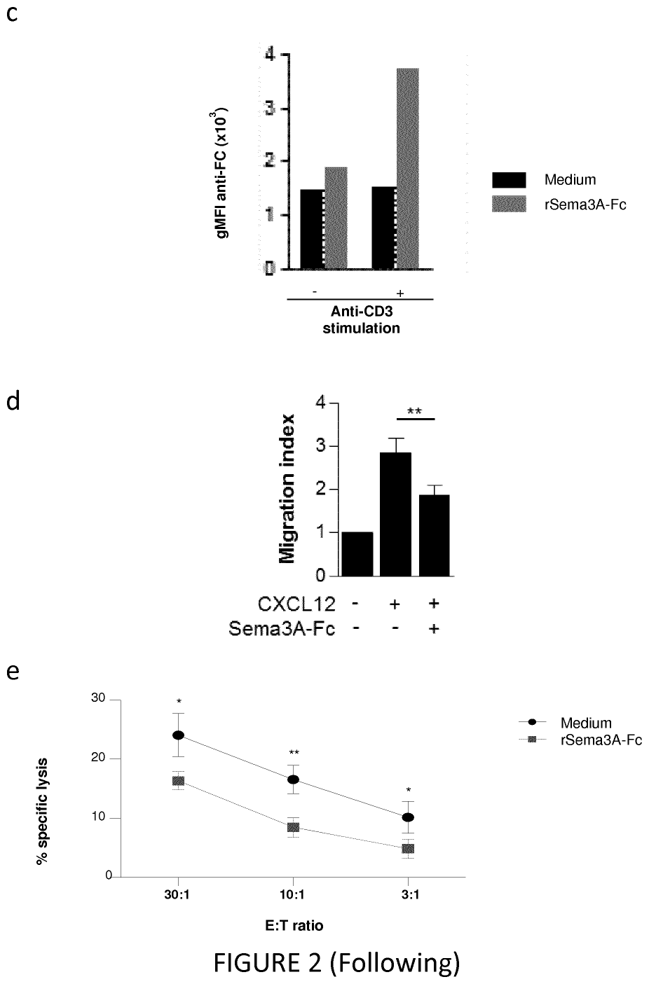

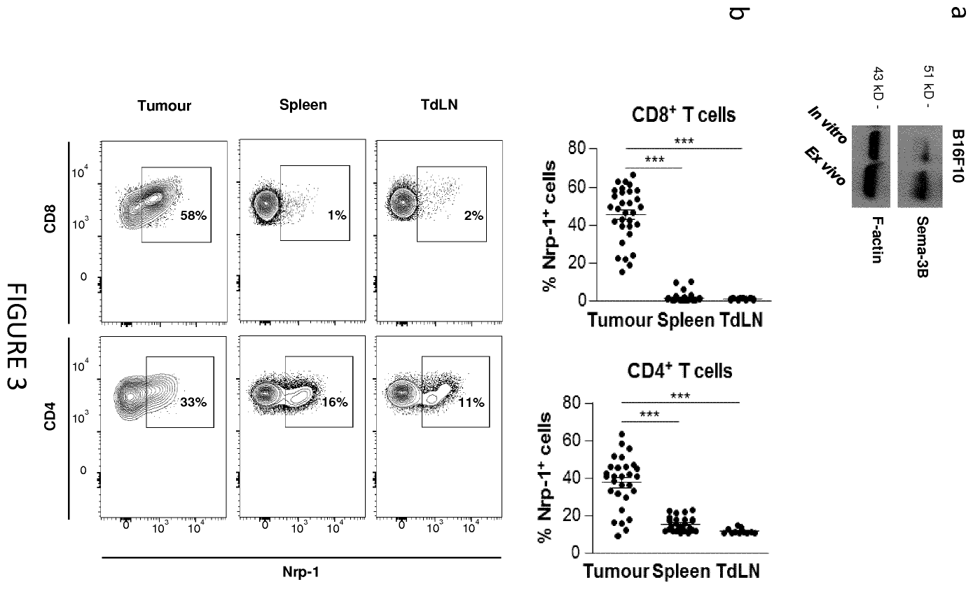

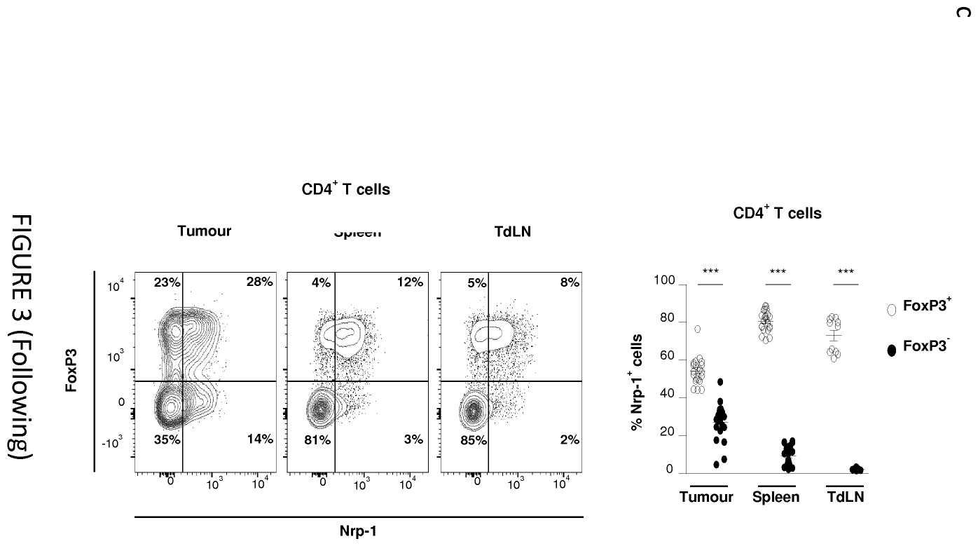

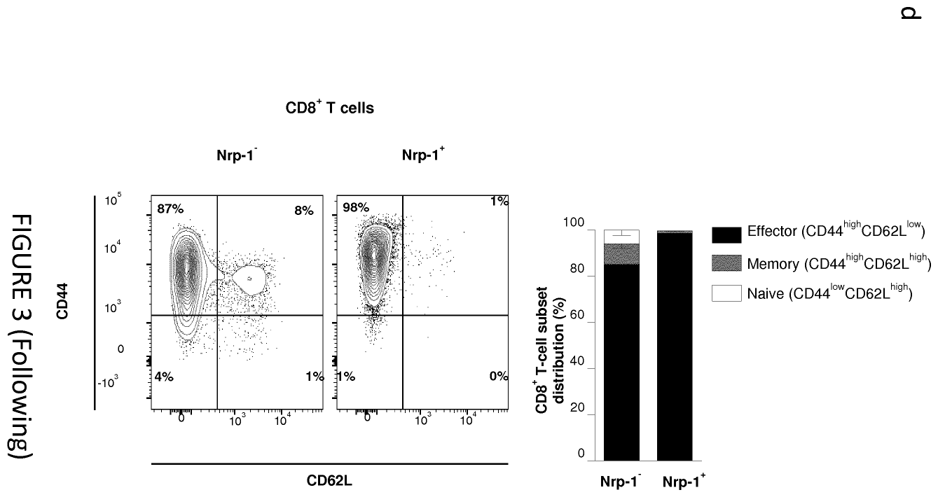

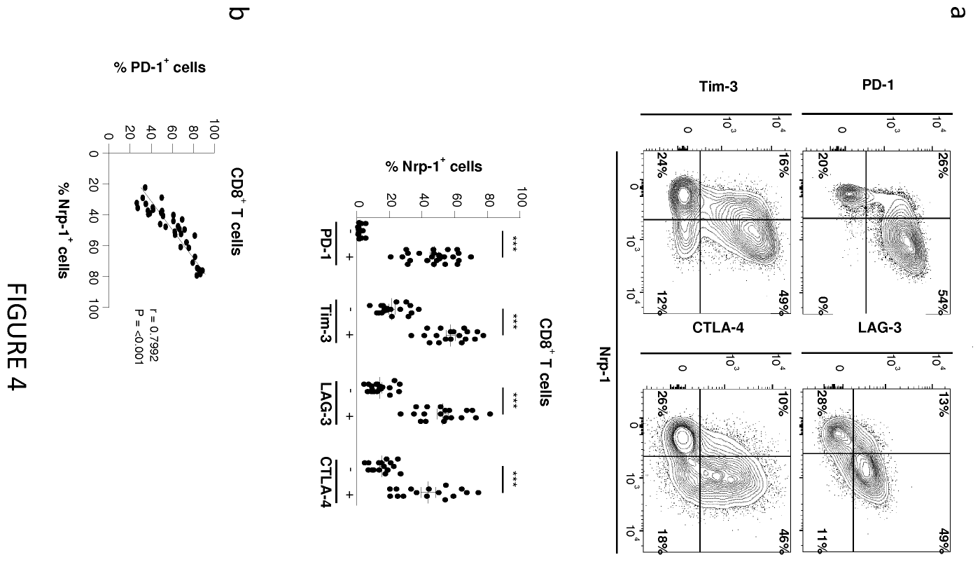

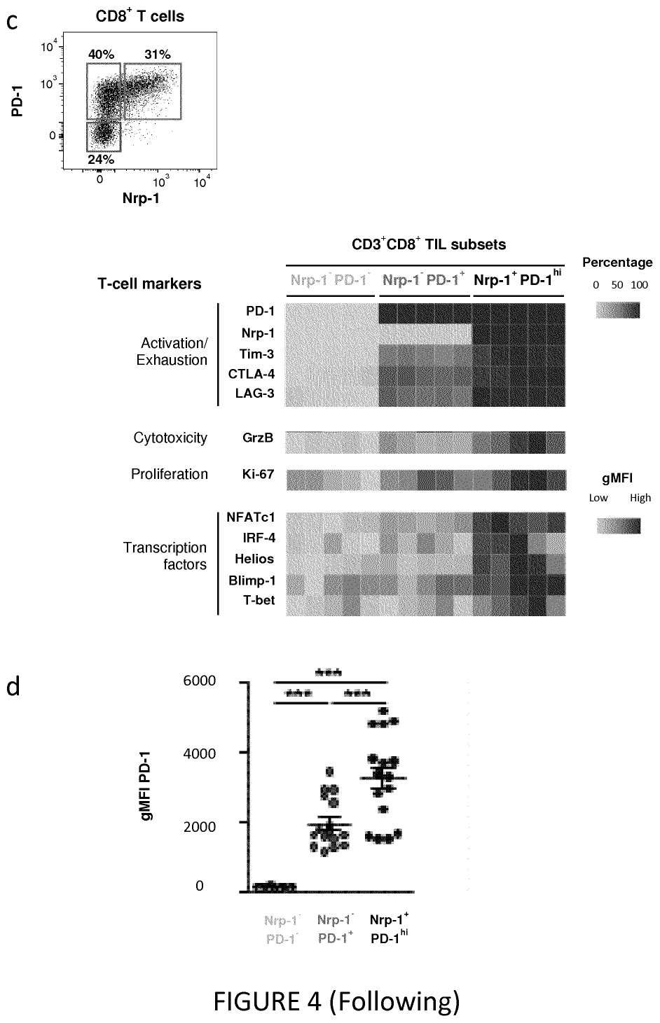

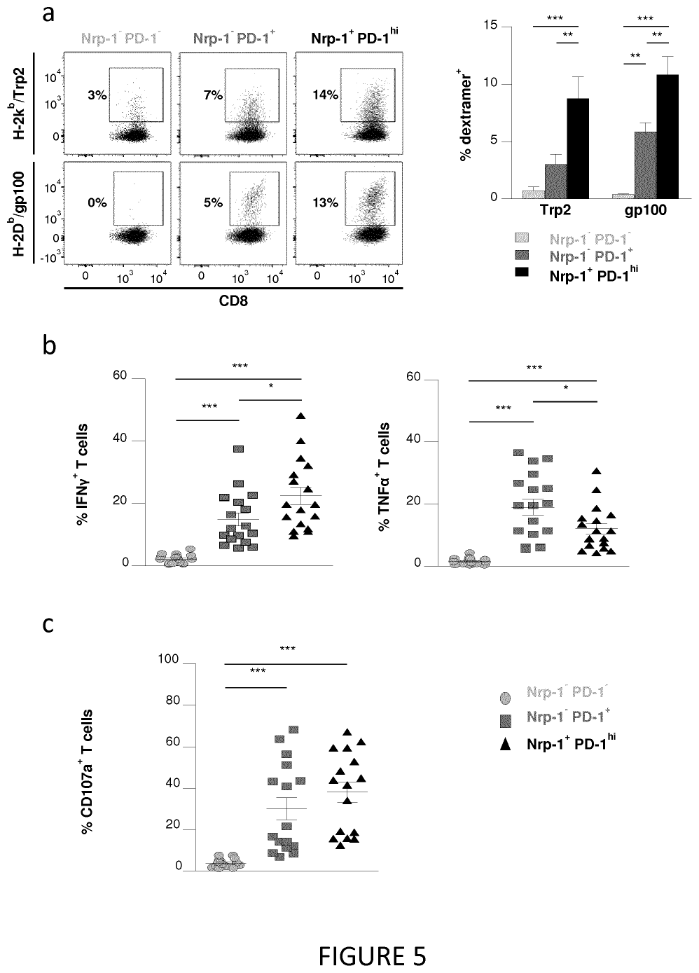

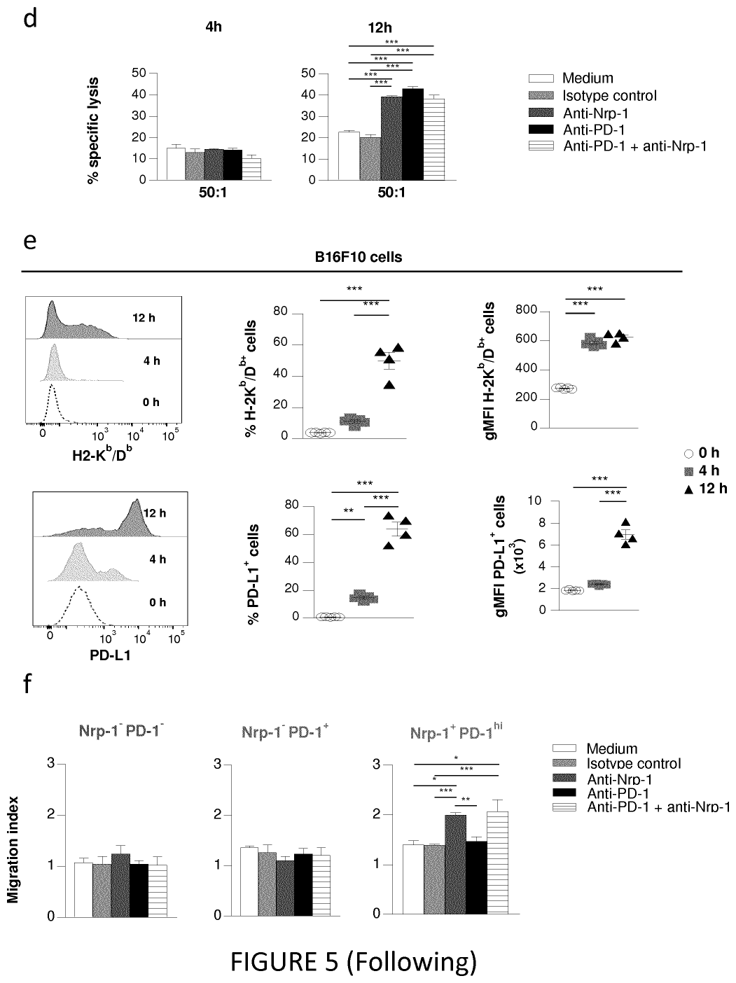

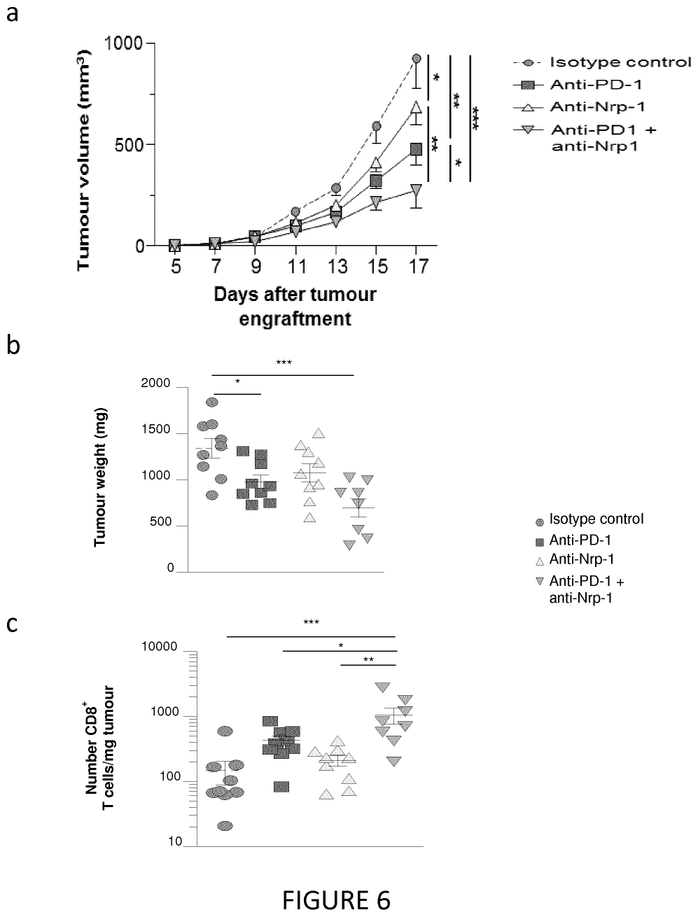

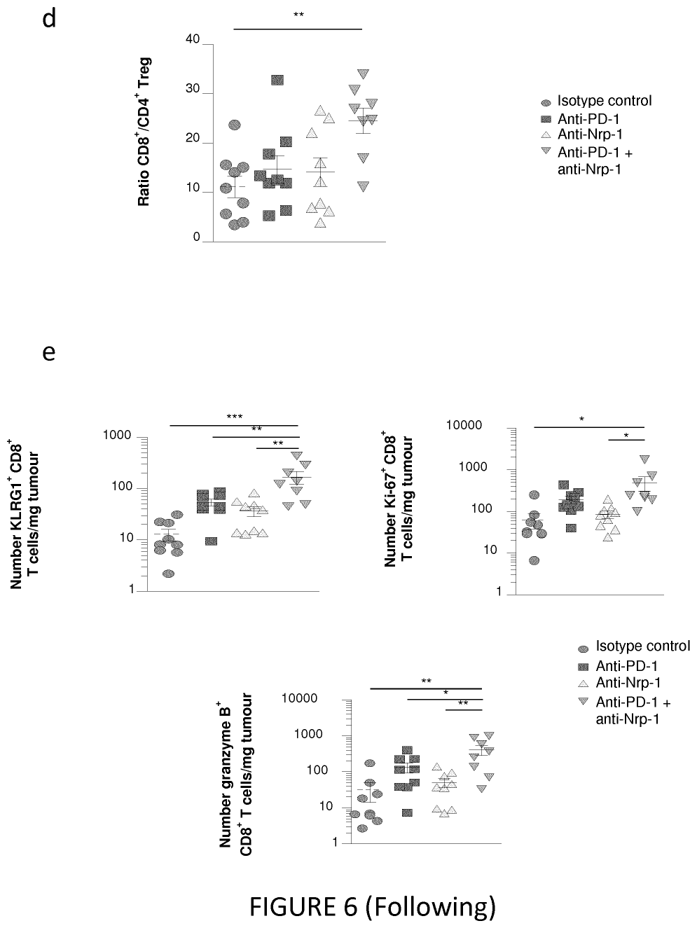

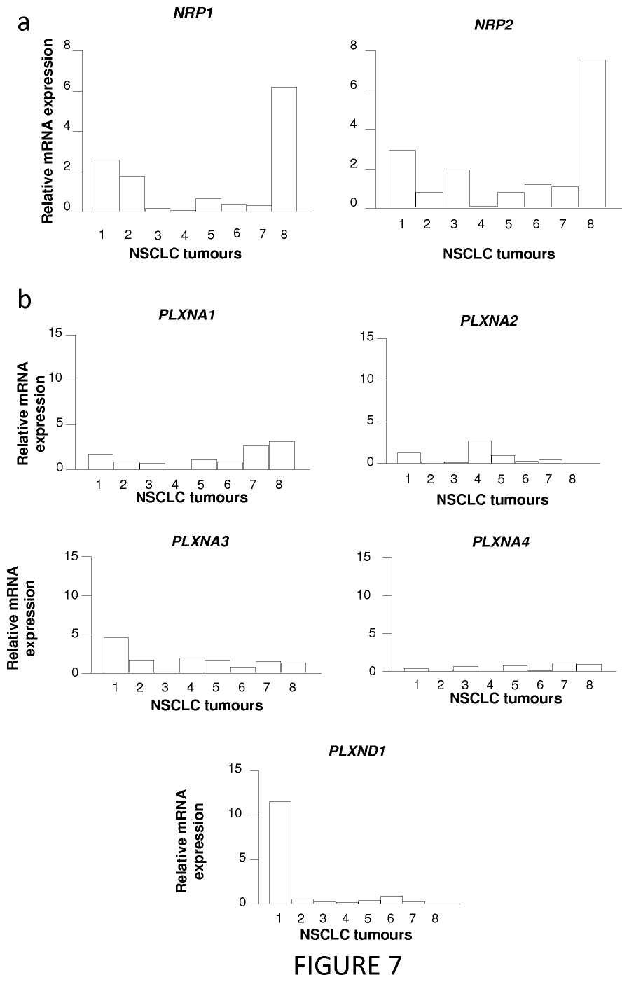

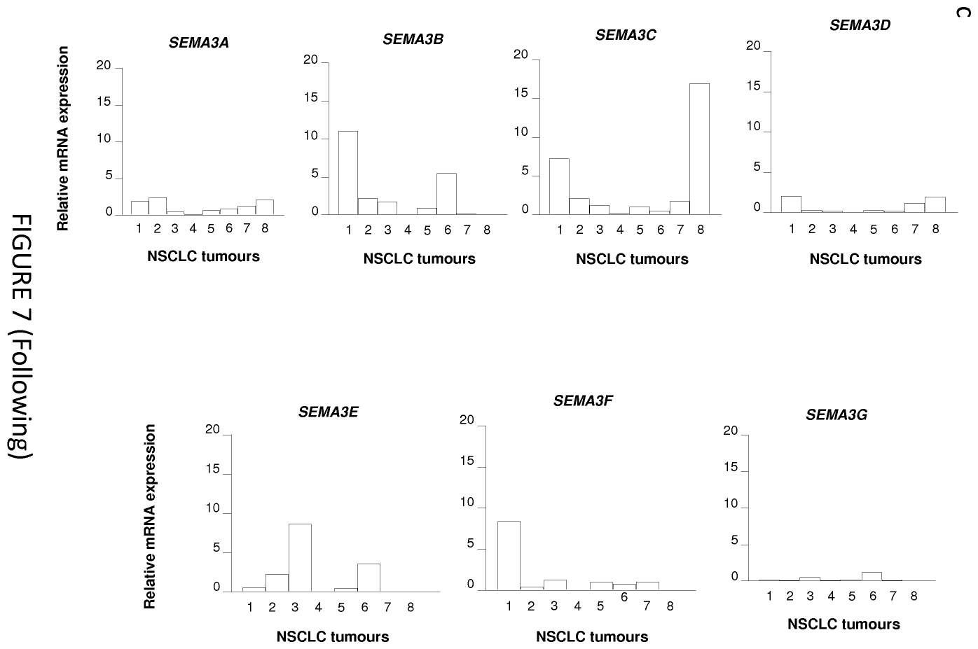

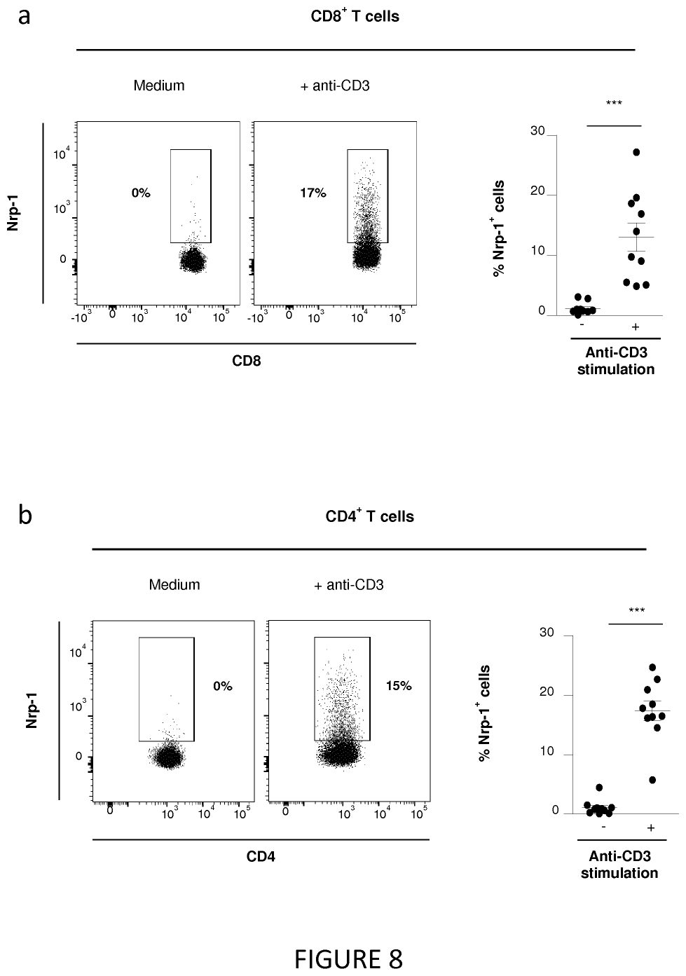

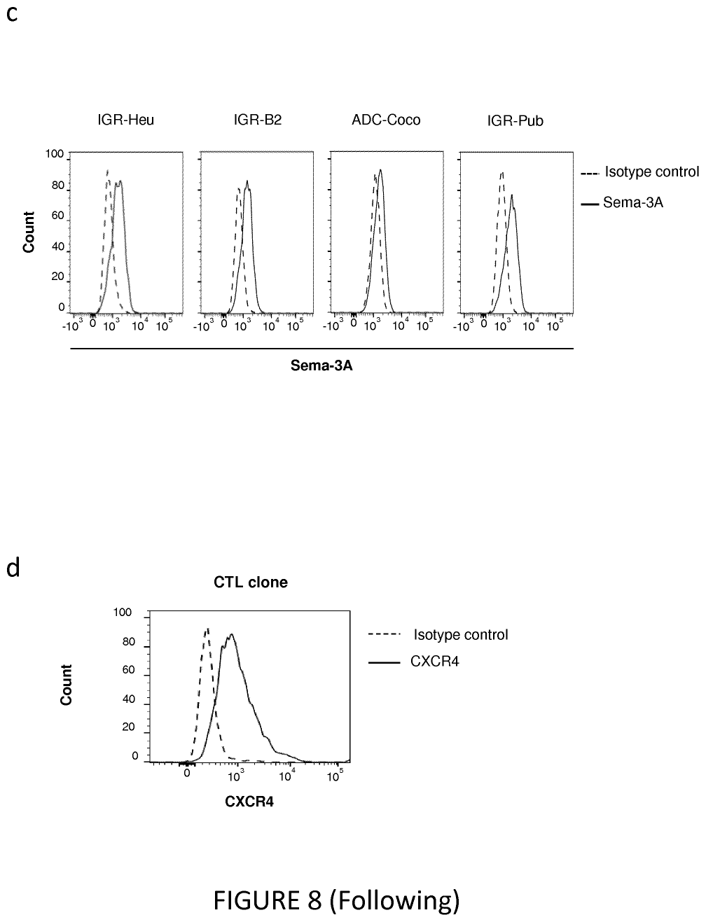

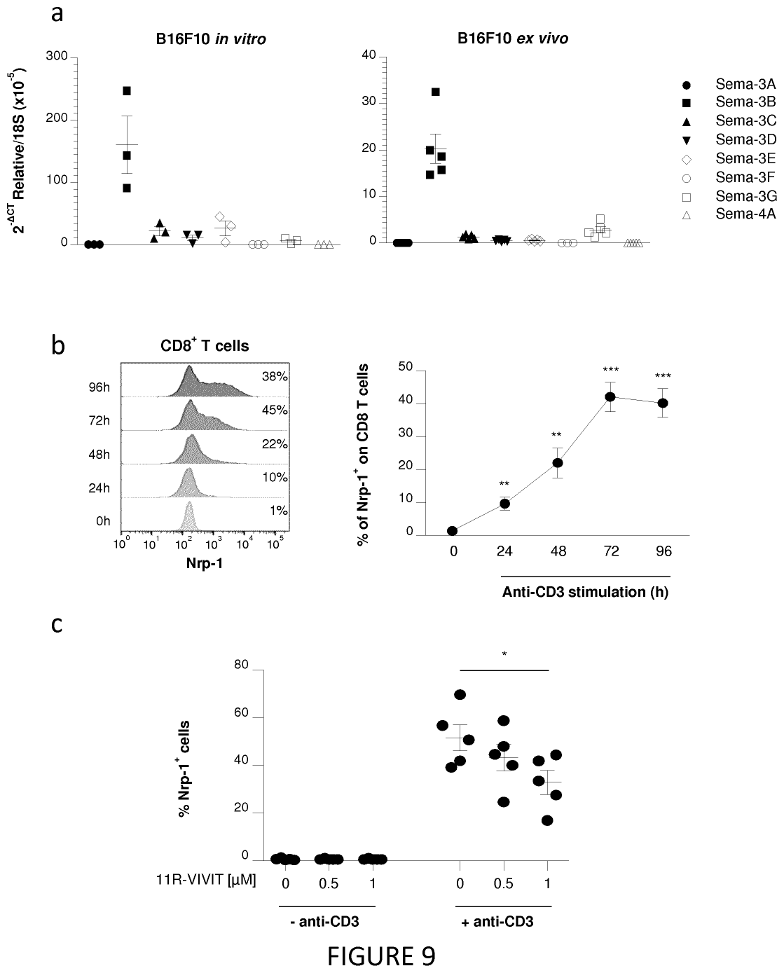

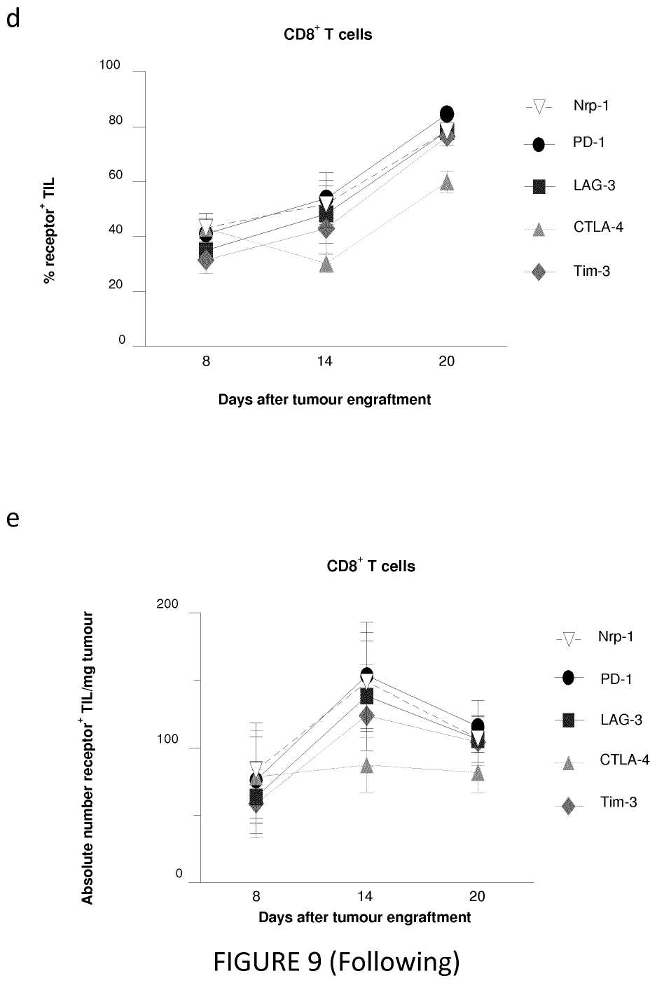

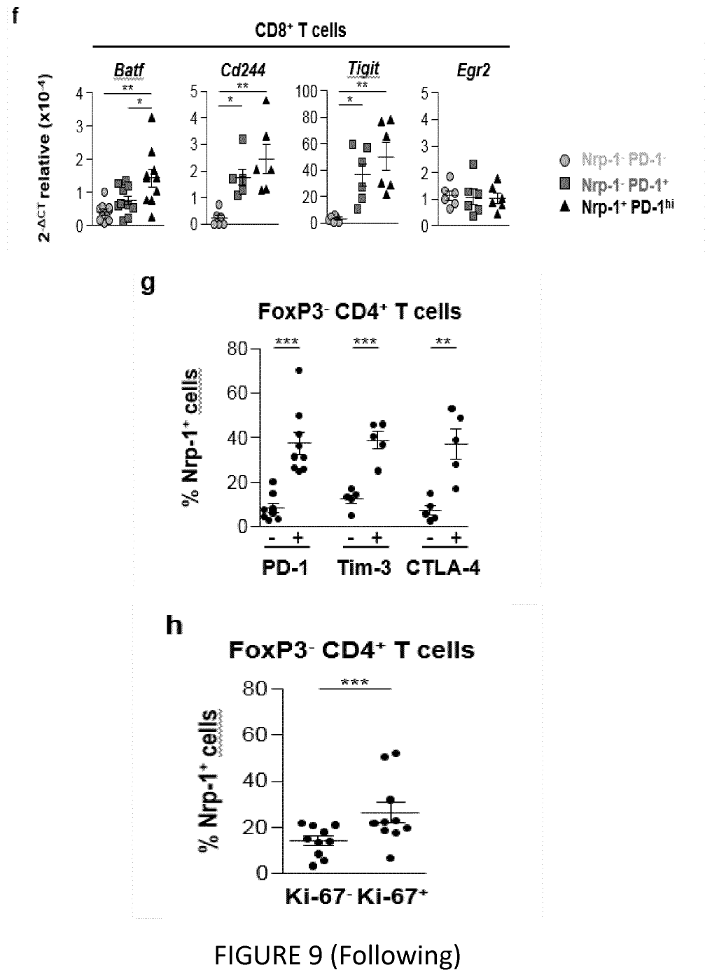

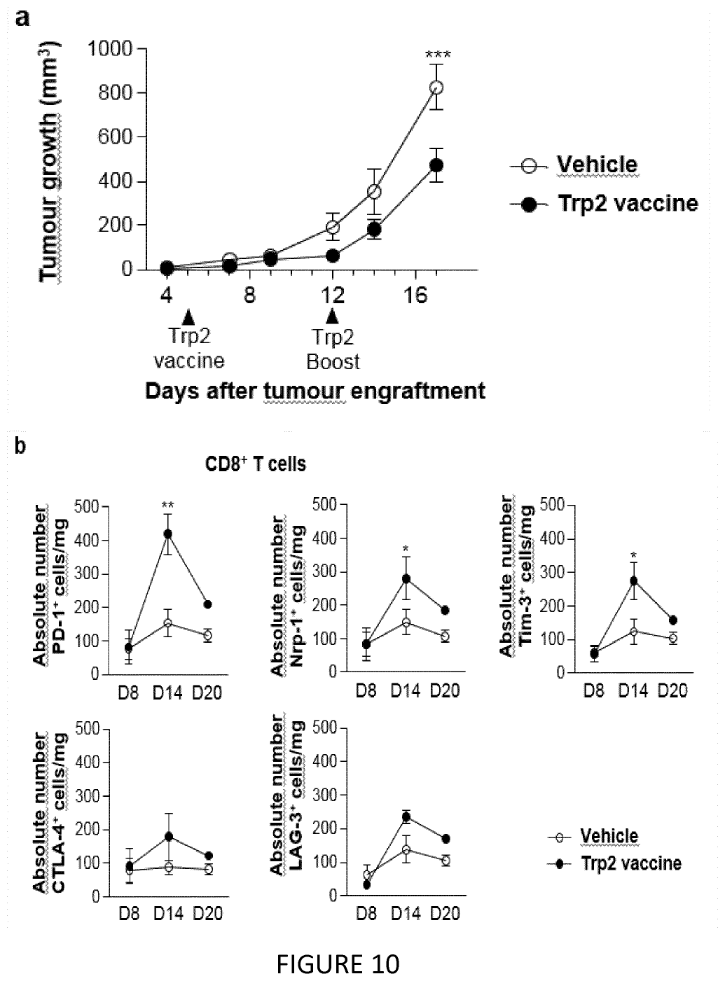

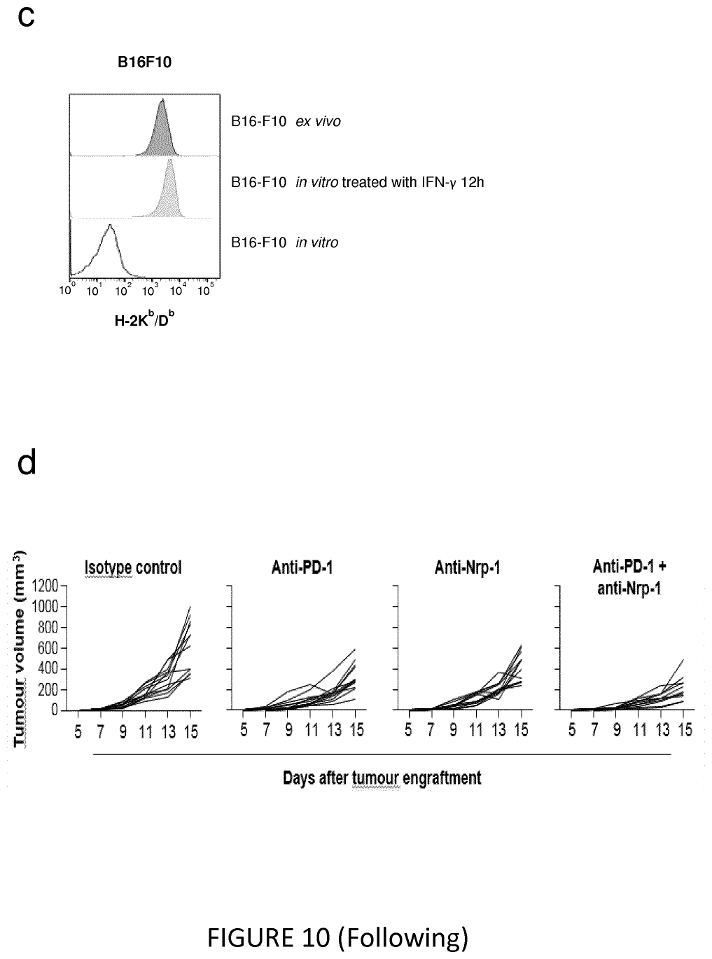

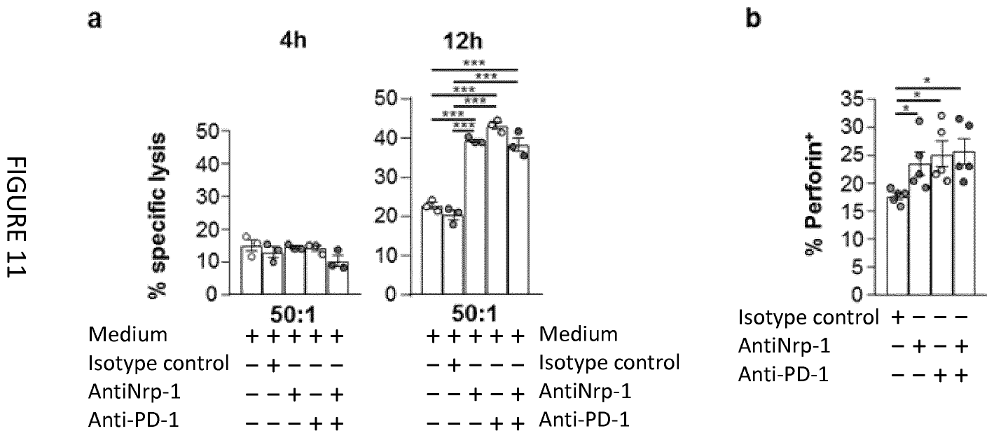

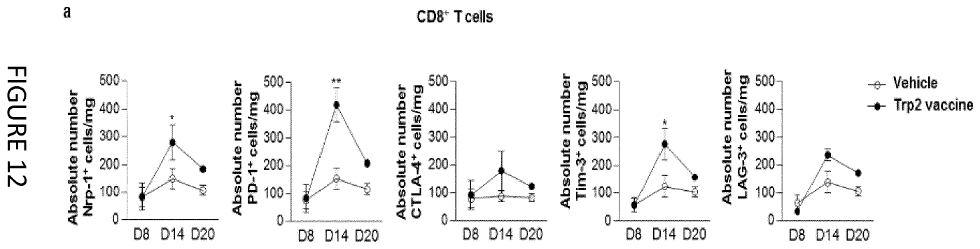

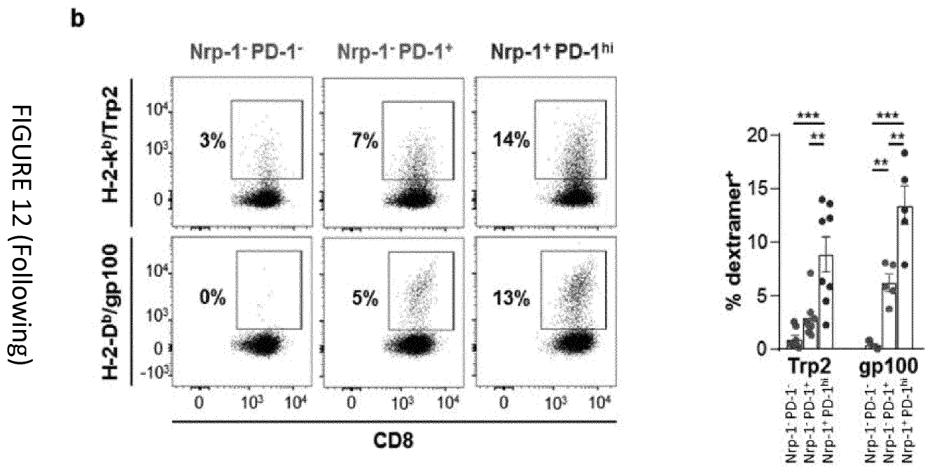

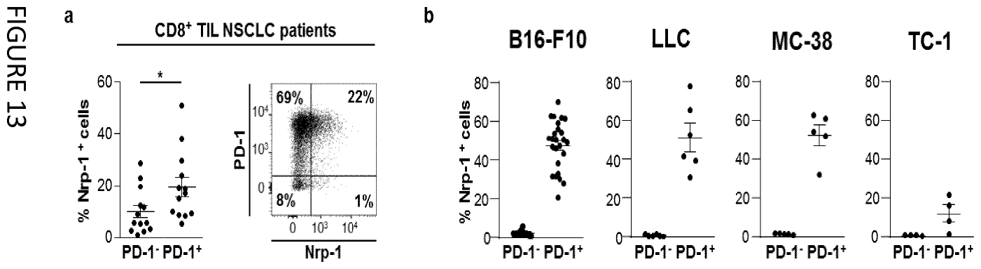

Inventors next evaluated the expression of Nrp-1 protein in freshly isolated TIL from 28 NSCLC tumors. Immunofluorescence analyses showed that Nrp-1 was expressed on a subset of human lung CD3⁺ TIL. In contrast, parallel analysis of peripheral blood lymphocytes (PBL) from lung cancer patients and healthy donors (HD) showed that circulating T cells did not express the receptor (FIG. 1a). Expression of Nrp-1 was observed on both CD8⁺ and CD4⁺ TIL, but with a higher frequency on CD8⁺ TIL (14.2±2.1% vs 8.4±0.9%). Moreover, expression of Nrp-1 correlated with the activation state of T lymphocytes, since it was more frequent on CD25⁺ and PD-1⁺ T cells from both CD8⁺ and CD4⁺ TIL subsets than on CD25⁻ and PD-1⁻ subsets (FIGS. 1b and c). Indeed, 70.1±8.3% of Nrp-1⁺ CD8⁺ TIL and 76.9±4.2% of Nrp-1⁺ CD4⁺ TIL also expressed high levels of PD-1 (PD-1^{hi}). Consistently, activation of HD PBL with immobilized anti-CD3 mAb induced expression of the protein (FIGS. 8a and b). In contrast, no correlation between Nrp-1 and Foxp3 expression was observed in CD4⁺ TIL, since equal percentages of Nrp-1⁺ T cells were found in both Foxp3⁺ and Foxp3⁻ subsets, which showed similar percentages of PD-1^{hi} cells (FIG. 1d). These results show that Nrp-1 is expressed on a subset of activated human CD4⁺ and CD8⁺ TIL displaying PD-1^{hi} status in NSCLC tumors.

Interaction of Human Nrp-1 with Sema-3A Impairs T-Cell Effector Functions In Vitro

Sema-3A, a secreted member of the Sema-3 family, is a well-known ligand of Nrp-1^{9,10}. As human CD8⁺ TIL expressed substantial amounts of Nrp-1, this raises the question as to whether its interaction with Sema-3A contributes to the T-cell dysfunction often observed in the tumor microenvironment. To evaluate this hypothesis, inventors first used the IGR-Pub lung adenocarcinoma cell line and autologous CTL clone P62 established from TIL of a NSCLC patient¹⁷. Initial studies indicated that the IGR-Pub cell line, as well as several other human lung cancer cell lines, and to a lesser extent, normal bronchial epithelial cell line 16HBE, produced Sema-3A, as detected by western blot (FIG. 2a) and intracellular immunofluorescence staining (FIG. 8c). In contrast, the P62 T-cell clone did not constitutively express Nrp-1. However, stimulation with plastic-coated anti-CD3 mAb induced strong Nrp-1 expression on the surface of P62 T cells, most of which also expressed IL-2 receptor subunit CD25 and inhibitory receptor PD-1 (FIG. 2b). Therefore, inventors used the anti-CD3-stimulated P62 CTL clone to examine the consequences of Sema-3A ligation to Nrp-1 on the migratory behaviour and cytotoxic activity of these T lymphocytes in vitro.

Previous immunofluorescence experiments indicated that P62 CTL bound rSema-3A-Fc (FIG. 2c) and expressed CXCL12 receptor CXCR4 (FIG. 8d). Consequently, P62 T cells were able to migrate toward a gradient of rCXCL12 chemokine in in vitro transwell assays (FIG. 2d). Inventors found that this T-cell migratory response was inhibited in the presence of rSema-3A-Fc (FIG. 2d). This impaired T-cell migration was correlated with inhibition by rSema-3A-Fc of the cytotoxic activity of the P62 CTL clone toward autologous IGR-Pub target cells (FIG. 2e). These results indicate that Nrp-1 triggering by its soluble ligand of the Sema-3 family Sema-3A negatively regulates effector functions of Nrp-1⁺ cytotoxic T cells, and shows a T-cell inhibitory receptor function for Nrp-1.

Nrp-1⁺ CD8⁺ T Cells Infiltrating Murine B16F10 Melanoma Display an Exhausted PD-1^{hi} State

To investigate the impact of Nrp-1 engagement with its ligand on CD8⁺ TIL functions in vivo, inventors used C57BL/6 mice engrafted with B16F10 melanoma cells, which express the Nrp-1 soluble ligand Sema-3B, as shown by RT-PCR analyses (FIG. 9a) and confirmed by western blot (FIG. 3a). At day 15, tumors were removed and TIL were isolated and analysed by flow cytometry. T cells from spleens and tumor-draining lymph nodes (TdLN) of the same mice were examined in parallel for Nrp-1 expression. Results showed that a large fraction of CD8⁺ TIL expressed Nrp-1 (45.5%±2.5), as opposed to spleens (1.9%±0.4) and TdLN (1.3%±0.1). The frequency of Nrp-1⁺ T cells was also higher in CD4⁺ TIL (37.7%±2.6), compared to spleens (15.7%±0.8) and TdLN (12%±0.4) (FIG. 3b). Moreover, while most Nrp-1⁺ CD4⁺ cells from spleens (80.5%±1.2) and TdLN (73%±2.7) expressed the Treg cell marker Foxp3, Nrp-1⁺ CD4⁺ TIL included both Foxp3⁺ (54.2%±1.7) and Foxp3⁻ (27.2%±2.2) T cells (FIG. 3c). It should be noted that most, if not all, CD8⁺ TIL displayed a CD44^{high} CD62L^{low} phenotype, characteristic of effector/memory T (T_{EM}) cells, especially when they co-expressed Nrp-1 (FIG. 3d). Notably, as in humans, expression of Nrp-1 was dependent on the activation state of T cells, since stimulation of CD3⁺ CD8⁺ mouse splenocytes with immobilized anti-CD3 induced Nrp-1 expression, which reached a plateau at 72 h (FIG. 9b) and was inhibited with the NFAT inhibitor 11R-VIVIT, indicating that the TCR signalling pathway was likely involved (FIG. 9c).

Inventors then focused on induction of Nrp-1 on CD8⁺ TIL during tumor progression. Expression of well-known inhibitory receptors PD-1, CTLA-4, Tim-3 and LAG-3 was monitored in parallel. Kinetic studies revealed that the percentage of Nrp-1⁺ CD8⁺ TIL increased during tumor growth, with similar induction of PD-1, LAG-3 and Tim-3 and, to a lesser extent, CTLA-4 (FIG. 9d). The absolute number of T cells (i.e. the number of TIL per mg of tumor) co-expressing these inhibitory receptors also increased during melanoma progression, and reached a peak at day 14 after tumor implantation (FIG. 9e). Remarkably, at day 14, all Nrp-1⁺ CD8⁺ TIL also expressed PD-1, and LAG-3, Tim-3 and CTLA-4 on most of them (FIG. 4a). This Nrp-1⁺ T-cell subset displayed a PD-1^{hi} profile (FIG. 4a), with a strong correlation between expressions of the two cell surface molecules (FIG. 4b). Heat map analyses showed that this Nrp-1⁺ PD-1^{hi} TIL subset included much higher percentages of T cells expressing other T-cell inhibitory receptors than the Nrp-1⁻ PD-1⁺ and Nrp-1⁻ PD-1⁻ subsets (FIG. 4c), with a much higher PD-1 geometric mean of fluorescence intensity (gMFI) than the Nrp-1⁻ PD-1⁺ subset (FIG. 4d). They also indicated that the Nrp-1⁺ PD-1^{hi} TIL subset included higher percentages of T cells expressing granzyme B and proliferation marker Ki67, as well as exhaustion-associated transcription factors NFATc1, IRF-4, Helios, Blimp-1 and T-bet, compared to the Nrp-1⁻ PD-1⁺ subset (FIG. 4c). Nrp-1⁺ PD-1^{hi} T cells also expressed high levels of Batf, Cd244 and Tigit transcripts, the products of which were associated with dysfunctional T-cell status, but not Egr2 mRNA (FIG. 9f)¹⁸. It should be noted that most Nrp-1⁺Foxp3⁻ CD4⁺ T cells were also found to express PD-1, Tim-3 and CTLA-4, as well as Ki-67 (FIG. 9g). These results indicate that Nrp-1 characterises a highly activated intratumoral CD8⁺ T-cell subset displaying PD-1^{hi} status and expressing several T-cell inhibitory receptors involved in immune suppression during cancer diseases.

Nrp-1 Typifies a Highly Activated Tumor-Specific CD8⁺ TIL Subset with Impaired Functional Activities

Next, inventors investigated the specificity and functionality of these Nrp-1⁺ PD-1^{hi} T cells. They first examined whether these lymphocytes were enriched with T cells specific to melanoma-associated antigens (MAA). To do so, C57BL/6 mice engrafted with B16F10 melanoma cells were vaccinated with Trp2 plus gp100 antigenic peptides, together with the poly(I:C) adjuvant. Antigenic specificity of intratumoral CD8⁺ TIL was then analysed with H-2K^{b}-Trp2 and H-2D^{b}-gp100 dextramers. Expression of Nrp-1 and PD-1 molecules on these TIL subpopulations was also monitored. Initial experiments showed that vaccinated mice more efficiently controlled tumor growth than unvaccinated mice (FIG. 10a). Moreover, at day 14 after melanoma cell transplantation, tumors from vaccinated mice showed increased absolute numbers of CD8⁺ TIL expressing PD-1, Nrp-1 and Tim-3 and, to a lesser extent, CTLA-4 and LAG-3 (FIG. 10b). More importantly, the percentage of Trp2-specific and gp100-specific CD8⁺ TIL in the Nrp-1⁺ PD-1^{hi} T-cell subset was over two-fold higher (8.8%±1.6 and 10.4%±2.6, respectively) than in Nrp-1⁻ PD-1⁺ (2.9%±0.7 and 5.6%±1.2, respectively) TIL (FIG. 5a). In contrast, the Nrp-1⁻ PD-1⁻ TIL subset showed very low percentages of MAA-specific T cells (0.9%±0.3 and 0.5%±0.3, respectively). Inventors' results also revealed that ex vivo stimulation of Nrp-1⁺ PD-1^{hi} TIL with autologous tumor cells induced higher percentages of IFNγ-producing T cells than in Nrp-1⁻ PD-1⁻ and the Nrp-1⁻ PD-1⁺ TIL subsets (FIG. 5b). Much stronger production of TNFα was also found with Nrp-1⁺ PD-1^{hi} CD8⁺ TIL compared to Nrp-1⁻ PD-1⁻ T cells, but in this case, percentages of TNFα-producing T cells remained lower than in the Nrp-1⁻ PD-1⁺ TIL population. This could be explained by a more advanced exhaustion stage of the Nrp-1⁺ PD-1^{hi} CD8⁺ TIL subset than the Nrp-1⁻ PD-1⁺ TIL subset as previously suggested during chronic viral infection¹⁹. Inventors then further assessed tumor-reactivity of Nrp-1⁺ PD-1^{hi} and Nrp-1⁻ PD-1⁺ TIL by measuring CD107a surface expression, a marker usually used to evaluate the degranulation capacity of CTL. Their results showed that ex vivo stimulation of TIL with the cognate B16F10 cell line induced much higher percentages of CD107a⁺ cells among Nrp-1⁺ PD-1^{hi} and Nrp-1⁻ PD-1⁺ TIL populations than did the Nrp-1⁻ PD-1⁻ T-cell subset (FIG. 5c).

Next, inventors analysed the cytotoxic activity of CD8⁺ TIL toward autologous melanoma cells in the absence or presence of neutralizing anti-Nrp-1 and/or anti-PD-1 mAb, with an isotype-matched mAb as negative control. A 4 h chromium (Cr⁵¹) release assay revealed that CD8⁺ TIL were poorly effective in killing B16F10 tumor cells, whether blocking mAb were present or not (FIG. 5d). In contrast, in a 12 h cytotoxicity assay, anti-Nrp-1 mAb, anti-PD-1 mAb, or a combination of both, strongly increased T-cell-mediated lysis. Notably, a combination of anti-Nrp-1 plus anti-PD-1 did not further increase target cell killing, suggesting that cytotoxicity is mainly mediated by the Nrp-1⁺ PD-1^{hi} TIL subset that includes most tumor-specific effector T cells (FIG. 5d). This cytotoxicity was correlated with upregulation of MHC-I molecules on the tumor cell surface (FIG. 5e). Indeed, parallel experiments in which inventors monitored H2-K^{b} and H2-D^{b} expression, as well as PD-L1, on B16F10 cells co-cultured for 12 h with CD8⁺ TIL, showed increase of the three molecules expression, a phenomenon in which IFNγ secreted by T cells was likely involved. Consistently, inventors observed such an upregulation of H2-K^{b}/-D^{b} surface expression after 12 h of stimulation of tumor cells with rIFNγ and on melanoma cells collected from in vivo tumor grafts (FIG. 10c).

To further investigate the influence of Nrp-1 on CD8⁺ TIL functions, inventors also performed migration assays in the presence of anti-Nrp-1 and/or anti-PD-1 neutralizing mAb of CD8⁺ TIL seeded for 24 h in upper chambers of transwell plates, with B16F10 target cells in the lower chambers. FACS analysis of T cells having migrated to the lower chambers in control conditions showed no difference between Nrp-1⁻ PD-1⁻, Nrp-1⁻ PD-1⁺ and Nrp-1⁺ PD-1^{hi} T-cell subsets (FIG. 5f). However, when TIL were pre-incubated with anti-Nrp-1 neutralizing mAb, the migration index of Nrp-1⁺ PD-1^{hi} T cells was strongly increased. Whatever the TIL subset, no effect was observed with anti-PD-1 alone. Moreover, within the Nrp-1⁺ PD-1^{hi} T-cell subset, no further increase was observed with a combination of anti-Nrp-1 and PD-1 mAb. These results support the conclusion that Nrp-1 behaves as a true CD8⁺ T-cell inhibitory receptor in vitro to impair the effector functions of anti-tumor CD8⁺ TIL.

Therapeutic Nrp-1 Blockade Potentiates Tumor Growth Control by Anti-PD-1 In Vivo

The above experiments suggested that Nrp-1 is a novel immune checkpoint expressed by tumor-reactive T cells that could impair their functional activities following interaction with its ligand Sema-3B. To directly test this hypothesis in vivo and therefore investigate the potential of neutralizing Nrp-1 via immunotherapeutic approaches, C57Bl/6 mice were engrafted with B16F10 melanoma and treated at days 6, 8, 10, 12 and 14 with anti-Nrp-1, anti-PD-1, a combination of both, or an isotype control mAb. Both tumor volume and tumor weight were followed up. Results showed that intratumoral (i.t.) administration of anti-Nrp-1 mAb, or intraperitoneal (i.p.) injection of anti-PD-1 mAb, inhibited tumor growth as compared to control treatment (FIGS. 6a and 6b). Importantly, a combination of anti-Nrp-1 plus anti-PD-1 was clearly additive, with much better control of tumor progression (FIG. 6a, FIG. 10d). A strong reduction in tumor weight measured at the experiment endpoint was also observed (FIG. 6b).

To further assess mechanisms involved in the therapeutic effect of anti-Nrp-1 plus anti-PD-1 combination, inventors analysed CD8⁺ TIL from tumors of the different groups of mice used above. Results showed much larger CD8⁺ T-cell infiltrates in tumors obtained from mice treated with the anti-Nrp-1 plus anti-PD-1 combination than from mice treated with each mAb alone or the isotype control (FIG. 6c). Increased CD8⁺/CD4⁺ Treg ratios were also observed in this group of mice compared to control mice (FIG. 6d). These CD8⁺ TIL expressed much higher levels of terminally differentiated effector T-cell marker KLRG1, as well as increased levels of proliferation marker Ki-67 and of serine protease granzyme B (FIG. 6e). Overall, these results demonstrate that anti-Nrp-1 plus anti-PD-1 in vivo treatment enhances tumor infiltration by Nrp-1⁺ PD-1^{hi} CD8⁺ TIL, with increased proliferative and killing capacities, leading to strong tumor regression. They also further emphasize the therapeutic potential and benefits of the anti-Nrp-1/anti-PD-1 combination in cancer immunotherapy.

Nrp-1⁺ TIL for Adoptive T-Cell Transfer Immunotherapy.

Inventors' results showed that blocking Nrp-1 induces an increase in the cytotoxic potential of CD8⁺ TILs revealed by increased cytotoxicity toward autologous tumor cells (FIG. 11a) and perforin intracellular expression (FIG. 11b). Inventors also demonstrated that CD8⁺ TILs expressing Nrp-1 are enriched with tumor-antigen-specific T cells (cf. FIG. 12).

Since these Nrp-1⁺ CD8⁺ TILs are enriched with tumor-specific T cells, they can be isolated using an anti-Nrp-1 antibody, amplified ex vivo and then injected into the patient for an adoptive transfer therapy.

Combination of a Therapeutic Anti-Cancer Vaccine with Anti-Nrp-1 and Optionally with Other Immune Check-Point Inhibitor(s)

Therapeutic cancer vaccines result in increase in the number of CD8⁺ T cells expressing inhibitory receptors into the tumor: Nrp-1, PD-1, Tim-3, CTLA-4 and LAG-3 (cf. FIG. 12a). These TILs are also enriched with tumor antigen-specific T lymphocytes (the tumor antigen used in the vaccine). Therefore, combining the cancer vaccine with anti-Nrp-1 antibodies (in combination or not with additional immune check-point blockers (ICB)/inhibitors (ICI), such as anti-PD-1, anti-TIM-3, anti-LAG-3 and/or anti-CTLA-4) will optimize the antitumor immune response and the clinical benefit of the immunotherapy, the cancer vaccine allowing the amplification of tumor-specific T cells and the ICB reactivating the induced T cells. FIG. 12 shows that the number of Nrp-1⁺ CD8⁺ TILs is enriched among tumor-specific T cells (cf. T cells specific to the tumor antigen used in the cancer vaccine and identified with the dextramers in FIG. 12b).

Relative or Minimum Amount of CD8⁺Nrp1⁺ (PD-1⁺) TILs Required to Identify Relevant Populations.

Inventors' data showed that the expression of Nrp-1 on CD8⁺ TILs differs from one tumor to another in human subjects and according to the tumor histological type (mouse studies):
in human lung cancer, the mean percentage of Nrp-1⁺ TILs among CD8⁺ TILs is around 15%±5% (FIG. 13a). Therefore, inventors believe that patients with a relative percentage of Nrp-1⁺ PD-1⁺ CD8⁺ TILs among TILs of the tumor sample of the subject of 15%±5%, identify the responding population.
in mouse tumor models (FIG. 13b), this percentage is much higher: around 50% for melanoma (B16F10), LLC lung cancer and colon cancer (MC-38). However, in TC-1 lung cancer, it is much lower (around 10%).

### DISCUSSION

Nrp-1 is essential in axonal guidance, through its capacity to bind class 3 chemorepulsive semaphorin proteins^{9,20}, and in angiogenesis, via its interaction with VEGF and VEGF receptors (VEGFR)^{21,22}. Nrp-1 is also involved in cardiovascular and neuronal development, cell migration, immunity and cancer^{8,23,24}. Indeed, tumor cells frequently express semaphorins and their receptors neuropilins and plexins, which can regulate malignant cell behaviour and contribute to malignant potential²⁴. Nrp-1 is also highly expressed on tumor vasculature, functioning as a mediator of tumor initiation and progression, associated with poor clinical outcome⁸. For instance, high expression of Nrp-1 observed in lung cancer correlates with invasive capacity and short disease-free survival²⁵. Moreover, cancer cells often produce secreted members of the semaphorin family, including Sema-3A and Sema-3B, also contributing to tumor escape from the immune response^{24,26}. Inventors now report, in both human NSCLC tumors and murine B16F10 melanoma, that Nrp-1 delineates a particular subset of CD8⁺ TIL, enriched with tumor antigen-specific T lymphocytes, and also expressing high levels of the PD-1 inhibitory receptor. In particular, they show that Nrp-1 works as a negative regulator of antitumoral activities of this CTL subset, and that blocking of this receptor in vivo strongly improves tumor regression elicited by anti-PD-1 immunotherapy.

Multiple roles for neuropilins and semaphorins in the immune system have emerged in recent years^{27,28}. Sema-3A has been reported to inhibit primary human T-cell proliferation and cytokine production under anti-CD3 plus anti-CD28 stimulation²⁶. It also promotes T-cell apoptosis²⁹ and inhibits non-specific cytotoxic activity of NK cells in mixed lymphocyte cultures²⁶. The influence of Sema-3A on T-cell migration has also been studied. In particular, this chemorepulsive molecule has been reported to inhibit immune cell migration and response of human T cells to chemokine gradients^{30,31}. Chemorepulsive effects of Sema-3A on human thymocytes have been reported, as well as inhibition of T-cell migratory responses triggered by chemokine CXCL1232,33. Inventors found in the context of the present invention that soluble Sema-3A binds to Nrp-1 molecules expressed on the surface of human lung tumor-specific CTL and inhibits their migratory capacity to a gradient of CXCL12 chemokine. Importantly, their results also showed that TCR-mediated killing of autologous human lung cancer cells was inhibited in the presence of Sema-3A, highlighting an inhibitory role of the Nrp-1/Sema-3A axis in CTL functions.

Nrp-1 is usually not expressed by resting T cells. However, its expression is triggered after T-cell activation, suggesting that Nrp-1 is an additional T-cell activation biomarker³⁴. Regulation of Nrp-1 expression during adaptive immune responses is likely an essential element for understanding its physiological role. In this context, activated T cells derived from inflammatory environments were described as expressing this surface molecule¹⁶. Indeed, Nrp-1 was highly induced on CD8⁺ T lymphocytes engaging self-antigen, including human melanoma TIL. Inventor now show that Nrp-1 is co-expressed with CD25 and PD-1 on both CD4⁺ and CD8⁺ TIL from human NSCLC. Moreover, on CD8⁺ TIL, this expression is restricted to a PD-1^{hi} T-cell subset. Various populations of CD8⁺ TIL have been recently described based on PD-1 expression levels: negative (PD-1^{N}), intermediate (PD-1^{hi}) and high (PD-1^{T})³⁵. Interestingly, inventors found a very good correlation between PD-1 and Nrp-1 expression (see FIG. 4b). Thus, the aforementioned PD-1^{T} TIL subset likely corresponds to cells expressing the highest levels of PD-1 and Nrp-1 identified in the context of the present invention. This is a crucial and surprising discovery, as this subset has been recently reported to correspond to an exhausted TIL subset³⁵. Accordingly, in the present experiments, inventors found that Nrp-1⁺ PD-1^{hi} TIL express transcription factors Helios, Blimp-1, IRF-4 and NFATc1, associated with activation/exhaustion³⁶⁻³⁹. Co-expression of Nrp-1 and Helios on CD4⁺ T cells was also observed on TIL from human liver metastases of colorectal cancer¹⁵. Another characteristic of PD-1^{T} TIL is that they display high tumor recognition capacity and are predictive of responses of human NSCLC patients to PD-1 blockade³⁵. Likewise, inventors found, in B16F10 tumors, that a significant percentage of Nrp-1⁺ PD-1^{hi} CD8⁺ TIL strongly expressed granzyme B and cell proliferation marker Ki-67, and secreted high levels of IFNγ. Moreover, this Nrp-1⁺ PD-1^{hi} T-cell subpopulation was the most strongly enriched with MAA-specific CD8⁺ T lymphocytes. Inventors therefore assume that Nrp-1 can be used as a novel marker for identifying this important CD8⁺ tumor-reactive TIL population. Overall, these findings suggest that, in an anti-tumor immune response context, Nrp-1 is a very late T-cell activation marker and that its co-expression with high levels of PD-1 might be another characteristic of exhausted antigen-experienced CD8⁺ TIL.

Expression of CTLA-4, PD-1 and Tim-3 molecules was associated with T-cell exhaustion in chronic viral infections and tumor progression⁴⁰⁻⁴⁴. These T-cell inhibitory receptors are important for regulating immune responses in peripheral tissues and maintaining self-tolerance. Along the same lines, Nrp-1 is induced on tolerant self-reactive CD8⁺ T cells expressing known regulatory receptors¹⁶. In the present report, inventors show that Nrp-1 is often associated with a broad panel of inhibitory receptors, such as CTLA-4, Tim-3 and LAG-3, and characterizes a dysfunctional PD-1^{hi} CD8⁺ TIL subpopulation. This particular phenotype, characteristic of T cells infiltrating an inflamed tumor microenvironment, may further explain the paradox of tumor progression, despite the presence of an ongoing T-cell response toward malignant cells. Inventors also show here that anti-Nrp-1 neutralising mAb, in particular when associated with anti-PD-1, re-established ex vivo the functionality of these T cells, such as externalization of CD107a and TCR-mediated cytotoxicity toward the cognate tumor. Remarkably, anti-Nrp-1 also restore the migratory capacity of Nrp-1⁺ PD-1^{hi} T cells, likely by blocking the interaction of Nrp-1 with its ligands Sema-3A or Sema-3B. Anti-PD-1 blocking mAb have no such effect. Semaphorins have been reported to govern cell migration by regulating integrin-mediated adhesion and actin cytoskeleton⁴⁵. A similar process may occur in tumors to explain how Nrp-1 signalling specifically affects T-cell migratory potential, possibly downstream plexin-A, which forms stable heterodimers with Nrp-1 and acts as a signal-transducing module for the Sema-3/Nrp-1/Plxn-A complex at the plasma membrane⁴⁶.

It is becoming increasingly clear that Nrp-1 and its ligands Sema-3A/-3B play important roles during the effector phase of various immune processes, including anti-tumor T-cell responses. Inventors' work now show that Nrp-1 negatively influences CD8 T-cell immunity and responses to anti-PD-1 cancer immunotherapy. Indeed, their in vivo experiments have revealed that, like the anti-PD-1 blockade, anti-Nrp-1 immunotherapy is able to inhibit melanoma progression in C57BL/6 mice. Moreover, a combination of the two antibodies is more efficient at reducing tumor growth associated with enhanced tumor infiltration by CD8⁺ effector T cells, with an increase in the CD8⁺/CD4⁺ T-cell ratio. This is an important finding, since the limited success of anti-PD-1 cancer immunotherapy is often associated with weak tumor infiltration by specific CD8⁺ T cells⁴⁷′48. Concomitant blockade of Nrp-1 and PD-1 could remedy this “cold-tumor” situation, thus providing a new immunotherapeutic strategy for further improving specific immune responses during cancer disease.

### REFERENCES

1. Mrass, P. et al. Random migration precedes stable target cell interactions of tumor-infiltrating T cells. J Exp Med 203, 2749-2761 (2006).
2. Topalian, S. L., Drake, C. G. & Pardoll, D. M. Immune checkpoint blockade: a common denominator approach to cancer therapy. Cancer Cell 27, 450-461 (2015).
3. McGranahan, N. et al. Clonal neoantigens elicit T cell immunoreactivity and sensitivity to immune checkpoint blockade. Science 351, 1463-1469 (2016).
4. Phan, G. Q. et al. Cancer regression and autoimmunity induced by cytotoxic T lymphocyte-associated antigen 4 blockade in patients with metastatic melanoma. Proc Natl Acad Sci USA 100, 8372-8377 (2003).
5. Topalian, S. L. et al. Safety, activity, and immune correlates of anti-PD-1 antibody in cancer. N Engl J Med 366, 2443-2454 (2012).
6. Tumeh, P. C. et al. PD-1 blockade induces responses by inhibiting adaptive immune resistance. Nature 515, 568-571 (2014).
7. Rizvi, N. A. et al. Cancer immunology. Mutational landscape determines sensitivity to PD-1 blockade in non-small cell lung cancer. Science 348, 124-128 (2015).
8. Prud'homme, G. J. & Glinka, Y. Neuropilins are multifunctional coreceptors involved in tumor initiation, growth, metastasis and immunity. Oncotarget 3, 921-939 (2012).
9. Kolodkin, A. L. et al. Neuropilin is a semaphorin III receptor. Cell 90, 753-762 (1997).
10. He, Z. & Tessier-Lavigne, M. Neuropilin is a receptor for the axonal chemorepellent Semaphorin III. Cell 90, 739-751 (1997).
11. Yadav, M. et al. Neuropilin-1 distinguishes natural and inducible regulatory T cells among regulatory T cell subsets in vivo. J Exp Med 209, 1713-1722, S1711-1719 (2012).
12. Delgoffe, G. M. et al. Stability and function of regulatory T cells is maintained by a neuropilin-1-semaphorin-4a axis. Nature 501, 252-256 (2013).
13. Hansen, W. et al. Neuropilin 1 deficiency on CD4+Foxp3+ regulatory T cells impairs mouse melanoma growth. J Exp Med 209, 2001-2016 (2012).
14. Battaglia, A. et al. Neuropilin-1 expression identifies a subset of regulatory T cells in human lymph nodes that is modulated by preoperative chemoradiation therapy in cervical cancer. Immunology 123, 129-138 (2008).
15. Chaudhary, B. & Elkord, E. Novel expression of Neuropilin 1 on human tumor-infiltrating lymphocytes in colorectal cancer liver metastases. Expert Opin Ther Targets 19, 147-161 (2015).
16. Jackson, S. R., Berrien-Elliott, M., Yuan, J., Hsueh, E. C. & Teague, R. M. Neuropilin-1 expression is induced on tolerant self-reactive CD8⁺ T cells but is dispensable for the tolerant phenotype. PLoS One 9, e110707 (2014).
17. Dorothee, G. et al. Tumor-infiltrating CD4⁺ T lymphocytes express APO2 ligand (APO2L)/TRAIL upon specific stimulation with autologous lung carcinoma cells: role of IFN-alpha on APO2L/TRAIL expression and -mediated cytotoxicity. J Immunol 169, 809-817 (2002).
18. Williams, J. B. et al. The EGR2 targets LAG-3 and 4-1BB describe and regulate dysfunctional antigen-specific CD8⁺ T cells in the tumor microenvironment. J Exp Med 214, 381-400 (2017).
19. Wherry, E. J., Blattman, J. N., Murali-Krishna, K., van der Most, R. & Ahmed, R. Viral persistence alters CD8 T-cell immunodominance and tissue distribution and results in distinct stages of functional impairment. J virol 77, 4911-4927 (2003).
20. Chen, H., He, Z., Bagri, A. & Tessier-Lavigne, M. Semaphorin-neuropilin interactions underlying sympathetic axon responses to class III semaphorins. Neuron 21, 1283-1290 (1998).
21. Staton, C. A., Kumar, I., Reed, M. W. & Brown, N. J. Neuropilins in physiological and pathological angiogenesis. J Pathol 212, 237-248 (2007).
22. Roskoski, R., Jr. Vascular endothelial growth factor (VEGF) signaling in tumor progression. Crit Rev Oncol Hematol 62, 179-213 (2007).
23. Fujisawa, H. Discovery of semaphorin receptors, neuropilin and plexin, and their functions in neural development. J Neurobiol 59, 24-33 (2004).
24. Muratori, C. & Tamagnone, L. Semaphorin signals tweaking the tumor microenvironment. Adv Cancer Res 114, 59-85 (2012).
25. Hong, T. M. et al. Targeting neuropilin 1 as an antitumor strategy in lung cancer. Clin Cancer Res 13, 4759-4768 (2007).
26. Catalano, A. et al. Semaphorin-3A is expressed by tumor cells and alters T-cell signal transduction and function. Blood 107, 3321-3329 (2006).
27. Suzuki, K., Kumanogoh, A. & Kikutani, H. Semaphorins and their receptors in immune cell interactions. Nat immunol 9, 17-23 (2008).
28. Kumanogoh, A. & Kikutani, H. Immunological functions of the neuropilins and plexins as receptors for semaphorins. Nat Rev Immunol 13, 802-814 (2013).
29. Moretti, S. et al. Semaphorin3A signaling controls Fas (CD95)-mediated apoptosis by promoting Fas translocation into lipid rafts. Blood 111, 2290-2299 (2008).
30. Mendes-da-Cruz, D. A., Linhares-Lacerda, L., Smaniotto, S., Dardenne, M. & Savino, W. Semaphorins and neuropilins: new players in the neuroendocrine control of the intrathymic T-cell migration in humans. Exp physiol 97, 1146-1150 (2012).
31. Mendes-da-Cruz, D. A. et al. Developing T-cell migration: role of semaphorins and ephrins. FASEB journal 26, 4390-4399 (2012).
32. Lepelletier, Y. et al. Control of human thymocyte migration by Neuropilin-1/Semaphorin-3A-mediated interactions. Proc Natl Acad Sci USA 104, 5545-5550 (2007).
33. Garcia, F. et al. Inhibitory effect of semaphorin-3A, a known axon guidance molecule, in the human thymocyte migration induced by CXCL12. J Leukoc Biol 91, 7-13 (2012).
34. Milpied, P. et al. Neuropilin-1 is not a marker of human Foxp3⁺ Treg. Eur J Immunol 39, 1466-1471 (2009).
35. Thommen, D. S. et al. A transcriptionally and functionally distinct PD-1(+) CD8(+) T cell pool with predictive potential in non-small-cell lung cancer treated with PD-1 blockade. Nat Med 24, 994-1004 (2018).
36. Bengsch, B. et al. Epigenomic-Guided Mass Cytometry Profiling Reveals Disease-Specific Features of Exhausted CD8 T Cells. Immunity 48, 1029-1045 e1025 (2018).
37. Shin, H. et al. A role for the transcriptional repressor Blimp-1 in CD8(+) T cell exhaustion during chronic viral infection. Immunity 31, 309-320 (2009).
38. Man, K. et al. Transcription Factor IRF4 Promotes CD8(+) T Cell Exhaustion and Limits the Development of Memory-like T Cells during Chronic Infection. Immunity 47, 1129-1141 e1125 (2017).
39. Martinez, G. J. et al. The transcription factor NFAT promotes exhaustion of activated CD8(+) T cells. Immunity 42, 265-278 (2015).
40. Trautmann, L. et al. Upregulation of PD-1 expression on HIV-specific CD8⁺ T cells leads to reversible immune dysfunction. Nat Med 12, 1198-1202 (2006).
41. Freeman, G. J., Wherry, E. J., Ahmed, R. & Sharpe, A. H. Reinvigorating exhausted HIV-specific T cells via PD-1⁻ PD-1 ligand blockade. J Exp Med 203, 2223-2227 (2006).
42. Kaufmann, D. E. et al. Upregulation of CTLA-4 by HIV-specific CD4⁺ T cells correlates with disease progression and defines a reversible immune dysfunction. Nature immunology 8, 1246-1254 (2007).
43. Blackburn, S. D., Shin, H., Freeman, G. J. & Wherry, E. J. Selective expansion of a subset of exhausted CD8 T cells by alphaPD-L1 blockade. Proc Natl Acad Sci USA 105, 15016-15021 (2008).
44. Fourcade, J. et al. Upregulation of Tim-3 and PD-1 expression is associated with tumor antigen-specific CD8+ T cell dysfunction in melanoma patients. J Exp Med 207, 2175-2186 (2010).
45. Casazza, A., Fazzari, P. & Tamagnone, L. Semaphorin signals in cell adhesion and cell migration: functional role and molecular mechanisms. Adv Exp Med Biol 600, 90-108 (2007).
46. Takahashi, T. et al. Plexin-neuropilin-1 complexes form functional semaphorin-3A receptors. Cell 99, 59-69 (1999).
47. Herbst, R. S. et al. Predictive correlates of response to the anti-PD-L1 antibody MPDL3280A in cancer patients. Nature 515, 563-567 (2014).
48. Melero, I., Rouzaut, A., Motz, G. T. & Coukos, G. T-cell and NK-cell infiltration into solid tumors: a key limiting factor for efficacious cancer immunotherapy. Cancer Discov 4, 522-526 (2014).
49. Echchakir, H. et al. Evidence for in situ expansion of diverse antitumor-specific cytotoxic T lymphocyte clones in a human large cell carcinoma of the lung. Inter Immunol 12, 537-546 (2000).
50. Le Floc'h, A. et al. Alpha E beta 7 integrin interaction with E-cadherin promotes antitumor CTL activity by triggering lytic granule polarization and exocytosis. J Exp Med 204, 559-570 (2007).
51. Le Maux Chansac, B. et al. Potentiation of NK cell-mediated cytotoxicity in human lung adenocarcinoma: role of NKG2D-dependent pathway. Inter Immunol 20, 801-810 (2008).
52. Le Floc'h, A. et al. Minimal engagement of CD103 on cytotoxic T lymphocytes with an E-cadherin-Fc molecule triggers lytic granule polarization via a phospholipase Cgamma-dependent pathway. Cancer Res 71, 328-338 (2011).

1-18. (canceled)

19. An in vitro or ex vivo method for detecting CD8⁺ TILs capable of recognizing cancer cells, the method comprising a) providing a biological sample comprising TILs and b) determining whether said TILs express Nrp-1, the expression of Nrp-1 by the TILs indicating that said TILs are capable of recognizing cancer cells.

20. The in vitro or ex vivo method for detecting CD8⁺ TILs capable of recognizing cancer cells according to claim 19, wherein step b) comprises determining i) whether said TILs express Nrp-1, and further comprises determining ii) whether said TILs express one or several receptors selected from CTLA-4, Tim-3 and LAG-3 and/or iii) the PD-1⁺ status of said TILs.

21. The in vitro or ex vivo method for detecting Nrp-1⁺ CD8⁺ TILs capable of recognizing cancer cells according to claim 19, wherein the CD8⁺ TILs capable of recognizing cancer cells are PD-1^{T} TILs.

22. The in vitro or ex vivo method for detecting CD8⁺ TILs capable of recognizing cancer cells according to claim 20, wherein the presence of Nrp-1⁺ PD-1⁺ CD8⁺ TILs is indicative that said TILs are dysfunctional.

23. The in vitro or ex vivo method for detecting CD8⁺ TILs capable of recognizing cancer cells according to claim 19, wherein the cancer is a solid tumor, a carcinoma, a sarcoma or a blastoma.

24. The in vitro or ex vivo method for detecting CD8⁺ TILs capable of recognizing cancer cells according to claim 23, wherein the cancer is a carcinoma, a lung cancer, non-small cell lung cancer (NSCLC) or a melanoma.

25. The in vitro or ex vivo method for detecting CD8⁺ TILs capable of recognizing cancer cells according to claim 19, wherein the cancer is a hematological tumor, a lymphoma, a leukemia or a multiple myeloma.

26. An in vitro or ex vivo method for predicting the response of a human subject to a treatment of lung cancer combining anti-PD-1 and anti-Nrp-1 agents, the method comprising detecting CD8⁺ TILs recognizing cancer according to the method of claim 19, the presence, in the CD8⁺ TILs of a biological sample of the subject, of about 15±5% of Nrp-1⁺ PD-1V CD8⁺ TILs being indicative that the human subject responds to the treatment of cancer.

27. A method for activating PD-1⁺ CD8⁺ tumor-infiltrating T lymphocytes (TILs) anti-cancer cell effector functions and/or for enhancing cancerous tumor infiltration by PD-1⁺ CD8⁺ TILs having active anti-cancer cells effector functions, or for treating cancer, in a subject, wherein the method comprises a step of administering, to a subject having a cancer Nrp-1⁺ CD8⁺ tumor-infiltrating T lymphocytes (TILs) exhibiting anti-cancer cell effector functions and/or enhanced cancerous tumor infiltration ability, wherein said TILs have been obtained from the subject having a cancer and have been amplified ex vivo before the administration step.

28. The method according to claim 27, wherein Nrp-1⁺ CD8⁺ TILs are PD-1^{T} TILs.

29. The method according to claim 27, wherein the cancer is a solid tumor, a carcinoma, a sarcoma or a blastoma.

30. The method according to claim 29, wherein the cancer is a carcinoma, a lung cancer, non-small cell lung cancer (NSCLC) or a melanoma.

31. The method according to claim 27, wherein the cancer is a hematological tumor, a lymphoma, a leukemia or a multiple myeloma.

32. A method for treating cancer in a subject, wherein the method comprises a step of administering an antibody combination comprising an effective amount of i) a Nrp-1 neutralizing antibody and ii) an immunotherapeutic antibody to a subject having a cancer, said subject expressing Nrp-1⁺ CD8⁺ TILs or Nrp-1⁺ PD-1⁺ CD8⁺ TILs.

33. The method for treating cancer according to claim 32, wherein the immunotherapeutic antibody is selected from a PD-1 neutralizing antibody, a TIM-3 neutralizing antibody, a LAG-3 neutralizing antibody and a CTLA-4 neutralizing antibody.

34. The method for treating cancer according to claim 32, wherein the immunotherapeutic antibody is a PD-1 neutralizing antibody.

35. The method for treating cancer according to claim 32, wherein the method further comprises a step of administering Nrp-1⁺ CD8⁺ TILs to the subject and wherein said TILs have been obtained from the subject having a cancer and have been amplified ex vivo before the administration step.

36. The method for treating cancer according to claim 32, wherein the cancer is a solid tumor, a carcinoma, a sarcoma or a blastoma.

37. The method for treating cancer according to claim 36, wherein the cancer is a carcinoma, a lung cancer, non-small cell lung cancer (NSCLC) or a melanoma.

38. The method for treating cancer according to claim 32, wherein the cancer is a hematological tumor, a lymphoma, a leukemia or a multiple myeloma.
